(19) United States
(12) Patent Application Publication
(10) Pub. No.: US 2019/0160005 A1
(43) Pub. Date: May 30, 2019

(54) Method of Preparing Solid Dispersions of Active Pharmaceutical Ingredients

(71) Applicant: Biophore India Pharmaceuticals PVT. Ltd., Hyderabad (IN)

(72) Inventors: Manik Reddy PULLAGURLA, Hyderabad (IN); Bhaskar Reddy PITTA, Hyderabad (IN); Rajesham BOGE, Hyderabad (IN); Suresh Babu NAMANA, Hyderabad (IN); Jagadeesh Babu RANGISETTY, Hyderabad (IN)

(21) Appl. No.: 16/199,255

(22) Filed: Nov. 26, 2018

(30) Foreign Application Priority Data
Nov. 24, 2017 (IN) 201741042228
Dec. 18, 2017 (IN) 201741045413
Dec. 18, 2017 (IN) 201741045418
Feb. 5, 2018 (IN) 201841004285
Feb. 5, 2018 (IN) 201841004286
Feb. 15, 2018 (IN) 201841005760
Apr. 25, 2018 (IN) 201841015635
Aug. 25, 2018 (IN) 201841031895

(51) Int. Cl. A61K 9/10; A61K 31/194; A61K 31/095; A61K 31/4025; A61K 31/69; A61K 31/496; A61K 31/513; A61K 31/519; A61K 31/417; A61K 31/55; A61K 47/10; A61K 47/40; A61K 47/38; A61K 47/32; A61K 47/26; A61K 47/22

(52) U.S. Cl. CPC A61K 9/10; A61K 9/19; A61K 31/095; A61K 31/4025; A61K 31/69; A61K 31/496; A61K 31/513; A61K 31/519; A61K 31/417; A61K 31/55; A61K 47/10; A61K 47/40; A61K 47/38; A61K 47/32; A61K 47/26; A61K 47/22; A61K 31/194

(57) ABSTRACT

The present invention provides solid dispersions of active pharmaceutical ingredients (APIs). It further relates to a process for the preparation of solid dispersions of active pharmaceutical ingredients (APIs) using pharmaceutically acceptable excipients.

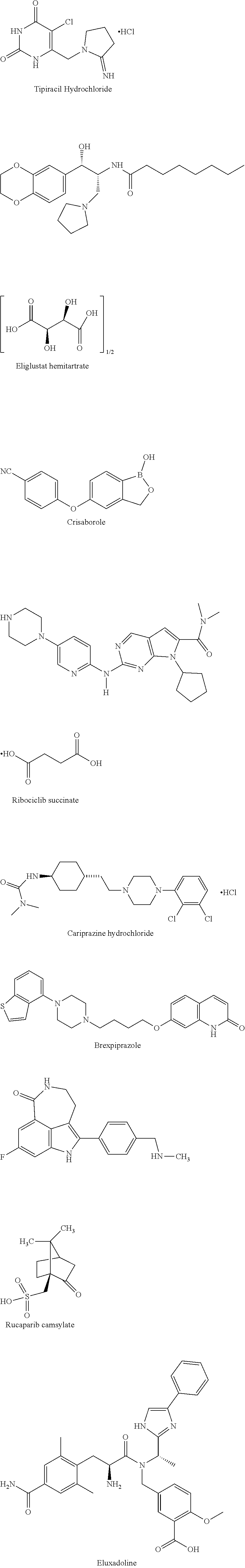

### CROSS REFERENCE

This application claims multiple priorities from Indian Patent application No. 201741042228 filed Indian Patent Office on Nov. 24, 2017; Indian Patent application No. 201741045413 filed Indian Patent Office on Dec. 18, 2017; Indian Patent application No. 201741045418 filed Indian Patent Office on Dec. 18, 2017; Indian Patent application No. 201841004285 filed Indian Patent Office on Feb. 5, 2018; Indian Patent application No. 201841004286 filed Indian Patent Office on Feb. 5, 2018; Indian Patent application No. 201841005760 filed Indian Patent Office on Feb. 15, 2018; Indian Patent application No. 201841015635 filed Indian Patent Office on Apr. 25, 2018; Indian Patent application No. 201841031895 filed Indian Patent Office on Aug. 25, 2018;

### BACKGROUND OF THE INVENTION

The following patents describes the solid dispersions of different active pharmaceutical ingredients (APIs)

U.S. Pat. No. 5,472,954 patent provides a method for enhancing the complexation formation of a lipophilic or water-labile active ingredient with a cyclodextrin.

U.S. Pat. No. 7,364,752 patent provides pharmaceutical composition comprising a solid dispersion of an HIV protease inhibitor in a water-soluble carrier.

U.S. Pat. No. 7,713,548 discloses the amorphous solid dispersions specifically of 7-chloro-N,N,5-trimethyl-4-oxo-3-phenyl-3,5-dihydro-4H-pyridazino[4,5-b] indole-1-acetamide using suitable excipient comprising hydroxypropyl methylcellulose phthalate, cellulose acetate phthalate, hydroxypropyl methyl cellulose acetate succinate, and a polymethacrylate.

Still there is need to develop stable forms of active pharmaceutical ingredients (APIs). Hence, the present inventors hereby report stable amorphous solid dispersions of some active pharmaceutical ingredients (APIs) and the process to produce the same.

### OBJECTIVE OF THE INVENTION

Accordingly, one objective of the present invention is to provide solid dispersions of active pharmaceutical ingredients.

In another objective, the present invention provides process for the preparation of amorphous solid dispersions of active pharmaceutical ingredients using suitable pharmaceutically acceptable excipients.

Yet, in another objective of the present invention provides solid dispersions using different drying techniques comprising lyophilization or freeze drying, spray drying, agitated thin film drying (ATFD), solvent evaporation, air tray drier or the like.

Still in another embodiment, the present invention provides solid dispersion by solvent cooling method or by adding anti-solvent to precipitate the solid

### SUMMARY OF THE INVENTION

Accordingly, one aspect of the present invention is to provide solid dispersions of active pharmaceutical ingredients.

In another aspect, the present invention provides process for the preparation of solid dispersions, which comprises of the following steps:
a) providing a solution of active pharmaceutical ingredient in a solvent or mixture of solvents;
b) adding pharmaceutically acceptable excipient to step a) solution;
c) optionally, heating the reaction mixture to a suitable temperature;
d) optionally, cooling the reaction mixture to a suitable temperature;
e) removing the solvent by a suitable solvent evaporation method
f) isolating solid dispersion of active pharmaceutical ingredient and excipient in an amorphous form.

In another aspect, the present invention provides solid dispersions of active pharmaceutical ingredients using different solvent evaporation techniques which may comprises of lyophilization or freeze drying, spray drying, agitated thin film drying (ATFD), solvent evaporation or the like. further provides solid dispersions by using solvent cooling method or by adding anti-solvent precipitation method.

In another aspect, the solid dispersion obtained in the above process is amorphous.

Yet in another aspect, solid dispersions obtained by any of the above methods were analysed using X-ray powder diffraction methods and obtained graphs as shown in FIG. 1 to FIG. 72

### BRIEF DESCRIPTION OF THE DRAWINGS

FIG. 1: X-Ray powder diffraction pattern of solid dispersion of Crisaborole with sulfobutylether-β-cyclodextrin (SBECD) by example-1

FIG. 2: X-Ray powder diffraction pattern of solid dispersion of Eliglustat hemitartrate with sulfobutylether-β-cyclodextrin (SBECD) prepared by example 2

FIG. 3: X-Ray powder diffraction pattern of solid dispersion of Eliglustat hemitartrate with sulfobutylether-β-cyclodextrin (SBECD) prepared by example-4.

FIG. 4: X-Ray powder diffraction pattern of solid dispersion of Cariprazine hydrochloride with sulfobutylether-β-cyclodextrin (SBECD) by example-6.

FIG. 5: X-Ray powder diffraction pattern of solid dispersion of Eliglustat hemitartrate with hydroxypropyl beta cyclodextrin (HPBCD) by example-8

FIG. 6: X-Ray powder diffraction pattern of solid dispersion of Eliglustat hemitartrate with 2-hydroxypropyl-beta-cyclodextrin (HPBCD) by example-10

FIG. 7: X-Ray powder diffraction pattern of solid dispersion of Eliglustat hemitartrate with Υ-cyclodextrin by example-11 FIG. 8: X-Ray powder diffraction pattern of dispersion of Eliglustat hemitartrate with Υ-cyclodextrin by example-13.

FIG. 9: X-Ray powder diffraction pattern of solid dispersion of Tipiracil hydrochloride with Polyvinylpyrrolidone (PVP) prepared by example-15.

FIG. 10: X-Ray powder diffraction pattern of solid dispersion of Tipiracil hydrochloride with Polyvinylpyrrolidone (PVP) prepared by example-16.

FIG. 11: X-Ray powder diffraction pattern of solid dispersion of Crisaborole with polyvinylpyrrolidone (PVP) as prepared in example-17.

FIG. 12: X-Ray powder diffraction pattern of solid dispersion of Tipiracil hydrochloride with lactose prepared by example-18.

FIG. 13: X-Ray powder diffraction pattern of solid dispersion of Tipiracil hydrochloride with hydroxy propyl methyl cellulose (HPMC) prepared by example-19.

FIG. 14: X-Ray powder diffraction pattern of solid dispersion of Tipiracil hydrochloride with hydroxy propyl methyl cellulose (HPMC) prepared by example-21

FIG. 15: X-Ray powder diffraction pattern of solid dispersion of Tipiracil hydrochloride with hydroxy propyl methyl cellulose (HPMC) by example-23.

FIG. 16: X-Ray powder diffraction pattern of solid dispersion of Crisaborole with hydroxypropyl methyl cellulose (HPMC) prepared by example-25 FIG. 17: X-Ray powder diffraction pattern of solid dispersion of Tipiracil hydrochloride with maltodextrin IT-19 prepared by example-26 FIG. 18: X-Ray powder diffraction pattern of solid dispersion of Tipiracil hydrochloride with maltodextrin IT-19 prepared by example-27 FIG. 19: X-Ray powder diffraction pattern of solid dispersion of Ribociclib succinate with sulfobutylether-β-cyclodextrin (SBECD) prepared by example-28 FIG. 20: X-Ray powder diffraction pattern of solid dispersion of Ribociclib succinate with sulfobutylether-β-cyclodextrin (SBECD) prepared by example-29 FIG. 21: X-Ray powder diffraction (XPRD) pattern of solid dispersion of Eluxadoline with sulfobutylether-β-cyclodextrin (SBECD) prepared by example-31 FIG. 22: X-Ray powder diffraction (XPRD) pattern of solid dispersion of Ribociclib succinate with sulfobutylether-β-cyclodextrin (SBECD) prepared by example-33

FIG. 23: X-Ray powder diffraction (XPRD) pattern of solid dispersion of Ribociclib succinate with sulfobutylether-β-cyclodextrin (SBECD) prepared by example-34

FIG. 24: X-Ray powder diffraction (XPRD) pattern of solid dispersion of Cariprazine hydrochloride with sulfobutylether-β-cyclodextrin (SBECD) prepared by example-36

FIG. 25: X-Ray powder diffraction (XPRD) pattern of solid dispersion of Rucaparib camsylate with Sulfobutylether-β-cyclodextrin (SBECD) prepared by example-37

FIG. 26: X-Ray powder diffraction (XPRD) pattern of solid dispersion of Brexpiprazole and Sulfobutylether-β-cyclodextrin (SBECD) prepared by example-38

FIG. 27: X-Ray powder diffraction pattern of solid dispersion solid dispersion of Eluxadoline with hydroxypropyl beta cyclodextrin (HPBCD) prepared by example-39

FIG. 28: X-Ray powder diffraction pattern of solid dispersion of Eluxadoline (1) with hydroxypropyl beta cyclodextrin (HPBCD) prepared by example-40

FIG. 29: X-Ray powder diffraction pattern of solid dispersion of Ribociclib succinate with hydroxypropyl beta cyclodextrin (HPBCD) prepared by example-42

FIG. 30: X-Ray powder diffraction pattern of solid dispersion of Cariprazine hydrochloride with hydroxypropyl beta cyclodextrin (HPBCD) prepared by example-46

FIG. 31: X-Ray powder diffraction pattern of solid dispersion of Brexpiprazole and Hydroxypropyl-β-cyclodextrin (HPBCD) prepared by example-47

FIG. 32: X-Ray powder diffraction pattern of solid dispersion of Eluxadoline with Y cyclodextrin prepared by example-48

FIG. 33: X-Ray powder diffraction pattern of solid dispersion of Brexpiprazole (1) and maltodextrin prepared by example-49

FIG. 34: X-Ray powder diffraction pattern of solid dispersion of Eluxadoline with hydroxy propyl methyl cellulose (HPMC) prepared by example-50

FIG. 35: X-Ray powder diffraction pattern of solid dispersion of Ribociclib succinate with hydroxypropyl methyl cellulose (HPMC) prepared by example-52

FIG. 36: X-Ray powder diffraction pattern of solid dispersion of Ribociclib succinate with hydroxypropyl methyl cellulose (HPMC) prepared by example-53

FIG. 37: X-Ray powder diffraction pattern of solid dispersion of Cariprazine hydrochloride with hydroxypropyl methyl cellulose (HPMC) prepared by example-54

FIG. 38: X-Ray powder diffraction pattern of solid dispersion of Cariprazine hydrochloride with hydroxy propyl methyl cellulose acetate succinate (HPMC-AS) prepared by example-55

FIG. 39: X-Ray powder diffraction pattern of solid dispersion of Brexpiprazole and Hydroxypropyl methyl cellulose (HPMC) prepared by example-56

FIG. 40: X-Ray powder diffraction pattern of solid dispersion of Eluxadoline with hydroxypropyl cellulose (HPC) prepared by example-57

FIG. 41: illustrates X-Ray powder diffraction (XPRD) pattern of solid dispersion of Ribociclib succinate with hydroxypropyl cellulose (HPC) prepared by example-59

FIG. 42: X-Ray powder diffraction (XPRD) pattern of solid dispersion of Brexpiprazole and Hydroxypropyl cellulose (HPC) prepared by example-60

FIG. 43: X-Ray powder diffraction (XPRD) pattern of solid dispersion of Cariprazine hydrochloride with hydroxypropyl cellulose (HPC) prepared by example-61

FIG. 44: X-Ray powder diffraction (XPRD) pattern of solid dispersion of Cariprazine hydrochloride with microcrystalline cellulose (MCC) prepared by example-62

FIG. 45: X-Ray powder diffraction (XPRD) pattern of solid dispersion of Eluxadoline with polyvinylpyrrolidone (PVP) prepared by example-63

FIG. 46: illustrates X-Ray powder diffraction pattern of solid dispersion of Cariprazine hydrochloride with polyvinylpyrrolidone (PVP K-30) prepared by example-65

FIG. 47: X-Ray powder diffraction (XPRD) pattern of solid dispersion of Ribociclib succinate with polyvinylpyrrolidone (PVP K-30) prepared by example-66

FIG. 48: X-Ray powder diffraction (XPRD) pattern of solid dispersion of Rucaparib camsylate with polyvinylpyrrolidone-K-30 (PVP-K-30) prepared by example-67

FIG. 49: X-Ray powder diffraction pattern of solid dispersion of Brexpiprazole (1) and Polyvinylpyrrolidone K-30 (PVP K-30) prepared by example-68

FIG. 50: X-Ray powder diffraction pattern of solid dispersion of Crisaborole with hydroxypropyl methyl cellulose (HPMC) prepared by example-73

FIG. 51: X-Ray powder diffraction pattern of solid dispersion of Crisaborole with hydroxypropyl methyl cellulose (HPMC) as prepared in example-74

FIG. 52: X-Ray powder diffraction pattern of solid dispersion of Crisaborole with hydroxypropyl cellulose (HPC) prepared by example-75

FIG. 53: X-Ray powder diffraction pattern of solid dispersion of Crisaborole with polyvinylpyrrolidone (PVP) as prepared in example-76

### DETAILED DESCRIPTION OF THE INVENTION

As used in the present specification, the following words and phrases are generally intended to have the meanings as set forth below, except to the extent that the context in which they are used indicates otherwise.

The term “solid dispersion” refers to the dispersion of one or more active pharmaceutical ingredients in an inert agent in a solid state prepared by a different methods, including spray drying, Lyophilization, solvent evaporation and drying by using agitated tray drier.

The term “active pharmaceutical ingredient” or “API” refers to a substance intended to be used as a component of a drug, and to furnish pharmacological activity or other direct effect in the diagnosis, cure, mitigation, treatment, or prevention of disease, or to affect the structure or any function of the organism to which it is administered.

The term “excipient” refers to a substance other than active pharmaceutical ingredient (API) that are intentionally included in an approved drug delivery system or a finished drug product.

In one embodiment, the present invention provides solid dispersions of active pharmaceutical ingredient (API) selected from the group but not restricted to drugs which can be used to treat dermatitis, Gaucher disease, irrigatable bowel syndrome, depression, psychosis, cancer or the like. Preferably, Crisaborole, Eliglustat hemitartrate, Eluxadoline, Ribociclib succinate, Rucaparib camsylate, Tipiracil hydrochloride, Brexpiprazole, Cariprazine hydrochloride or the like.

The suitable pharmaceutically acceptable excipients used in the present invention to prepare mot stable solid dispersions were selected from a group comprising of, cyclodextrins, cellulosic derivatives, vinyl pyrrolidone polymers and co-polymers, sugar derivatives or the like. Cyclodextrins may comprise of α-cyclodextrins, β-cyclodextrins and Υ-cyclodextrins. Among these mostly β-cyclodextrins is currently the most common cyclodextrin in pharmaceutical formulations, which comprise of sulfobutylether-β-cyclodextrin (SBECD), hydroxypropyl beta cyclodextrin (HPBCD). Other cyclodextrins used often are Υ-cyclodextrin, maltodextrin or the like. Celluloses or cellulose derivatives used herein may be selected from a group comprising of methyl cellulose, ethyl cellulose, microcrystalline cellulose, carboxymethyl cellulose (CMC), hydroxyethyl cellulose (HEC), hydroxy propyl methyl cellulose (HPMC), hydroxypropyl cellulose (HPC), hydroxy propyl methyl cellulose acetate succinate (HPMC-AS), hydroxypropyl cellulose (HPC), cellulose acetate, cellulose acetate phthalate (CAP); vinyl pyrrolidone polymers and copolymers comprising of polyvinyl alcohol, polyvinylpyrrolidone-vinyl acetate, polyvinylpyrrolidone, polyvinylpyrrolidone K-30 (PVP K-30), polyvinylpyrrolidone K-60 (PVP K-60), polyvinylpyrrolidone K-90 (PVP K-90), Soluplus or the like; sugar derivatives comprising of lactose, mannose, saccharose, sorbitol, mannitol, xylitol or the like. Preferably, sulfobutylether-β-cyclodextrin (SBECD), hydroxypropyl beta cyclodextrin (HPBCD), Υ-cyclodextrin, maltodextrin, microcrystalline cellulose, hydroxy propyl methyl cellulose (HPMC), polyvinylpyrrolidone K-30 (PVP-K-30), sulfobutylether-β-cyclodextrin (SBECD), hydroxy propyl methyl cellulose (HPMC), lactose, hydroxypropyl cellulose (HPC) were used in the present invention.

In another embodiment, using cyclodextrins is more advantageous over other excipients due to α and β-cyclodextrins are highly water soluble than other polar solvents and thus increases the water solubility of several poorly water-soluble substances. Increase in solubility in turn, enhances the bioavailability of the active pharmaceutical ingredient. Υ-cyclodextrins is the fastest degradable compound, by α-amylases. Adsorption studies have shown that only 2-4% of cyclodextrins were adsorbed in the small intestines and remaining is degraded and used up as glucose. Hence, cyclodextrins when administered orally are less toxic.

In another embodiment, the present invention provides process for the preparation of solid dispersions of active pharmaceutical ingredients, which comprises of the following steps:
a) providing a solution of active pharmaceutical ingredient in a solvent or mixture of solvents;
b) adding pharmaceutically acceptable excipient to step a) solution;
c) optionally, heating the reaction mixture to a suitable temperature;
d) optionally, cooling the reaction mixture to a suitable temperature;
e) removing the solvent by a suitable solvent evaporation method
f) isolating solid dispersion of active pharmaceutical ingredient and excipient in an amorphous form.

In one embodiment, the starting material pharmaceutically active ingredient used in the present invention can be crystalline, amorphous, solvate, salt or free base.

In another embodiment, the molar ratio of the active pharmaceutical ingredient and the excipient in the admixture is a range of 1:0.3 to 1:20 and in some cases, the ratio ranges from 1:0.2.

In one embodiment, the suitable temperature used in the present invention in step c) ranges from 40-100° C., preferably from 50-65° C. and more preferably from 60-65° C.,

In another embodiment, the reaction mixture in step d) was cooled to 10-35° C., preferably to 25-30° C.

The suitable solvents used in the present invention was selected from a group comprising of protic, aprotic solvents or mixtures thereof. The protic solvents were selected from a group comprising of water, methanol, ethanol, isopropyl alcohol, isobutyl alcohol, dimethyl sulfoxide, or the like, preferably water, methanol and isopropyl alcohol were used in the present invention; the aprotic solvents were selected from a group comprising of acetone, acetonitrile, nitromethane, 1,4-dioxane, diethyl ether, dichloromethane, ethyl acetate, N, N-dimethylformamide, methyl tertiary butyl ether, hexane, butyl acetate cyclohexane, toluene, tetrahydrofuran or the like, preferably dichloromethane and cyclohexane were used in the present invention.

In one embodiment, the suitable techniques used for the isolation of solid dispersions are comprising of solvent evaporation, lyophilization or freeze drying, spray drying, agitated thin film drying (ATFD), air tray drying (ATD), cooling the solvent, adding anti-solvent to the reaction mixture and combination thereof

In one embodiment, solvent evaporation method involves the, dissolution of both active pharmaceutical ingredient and excipient in a solvent or mixture of solvents. Optionally heating the reaction mixture to get the clear solution and evaporating the solvent by rotatory evaporator under pressure and suitable temperature conditions. Optionally, the drying of the solid dispersions can be done by using air tray drier at normal atmospheric pressure. Solid dispersions of Brexpiprazole, Eliglustat hemitartrate, Eluxadoline, Rucaparib camsylate, Ribociclib succinate, Cariprazine hydrochloride were prepared by solvent evaporation method.

In another embodiment, lyophilization or freeze-drying apparatus used in the present invention can be selected from any of the various commercially available apparatus. The technique was proposed an alternative to the solvent evaporation. This method involves dissolving active pharmaceutical ingredient and excipient in a solvent or mixture solvents then heating the reaction mixture to get the clear solution. The obtained clear solution can be frozen and sublimed to obtain a lyophilized solid dispersion. The details of the apparatus used in the present invention as mentioned below.

Operational Parameters:

Name of instrument: Lyomax Industrial Freeze Dryer

Inlet temperature: −40° C.

Vacuum Pressure: 30 to 10 torr

In another embodiment, spray drying method involves breaking up of liquid mixtures into small droplets and rapidly removing solvent from the mixture in a container (spray drying apparatus) that provides a strong driving force inducing evaporation of solvent from the droplets. The spray drying method used in the present invention involves mixing of active pharmaceutical ingredient and excipient in a solvent or mixture of solvents. Optionally heating the reaction mixture to obtain a clear solution. The obtained solution can be spray dried under the below mentioned conditions.

The, operating parameters for the spray drying method used were as discussed below

Name of instrument: Jay Instruments mini spray dryer

Aspirator: 70%

Feed rate: 10 mL/min

Inlet temperature: 65° C.,

Nitrogen Gas flow: 2 kg/cm²

In another embodiment, the agitated thin-film dryers (ATFDs) are used to produce dry free-flowing powder from slurry/solution-type feed. This method involves preparation of mixture of active pharmaceutical ingredient and excipient in a solvent or mixture of solvents. Optionally heating the reaction mixture to obtain a clear solution. The obtained solution can be dried by using commercially available agitated thin-film dryers can be used. The operating parameters for agitated thin film drier method (ATFD) used were as mentioned below:

Vacuum: 730 mm/Hg

Feed rate: 15 mL/min

Inlet temperature: 65° C.,

Condensed Temperature: −5° C.

In another embodiment, solvent cooling method involves mixing of the active pharmaceutical ingredient and excipient in a solvent or mixture of solvents. Optionally heating the reaction mixture to obtain a clear solution. Then cooling the reaction mixture to suitable temperature to precipitate the solid dispersion of the active ingredient. In another embodiment, anti-solvent precipitation method involves the addition of suitable solvent to a solution of active pharmaceutical ingredient and excipient at a suitable temperature to precipitate the solid. The obtained solid dispersion can be dried under vacuum.

In one embodiment, the suitable temperature used in the present invention for heating the reaction mixture ranges from 40-100° C., preferably from 50-65° C. and more preferably from 60-65° C., In another embodiment, cooling temperature was ranges from 10-35° C. and more preferably from 25-30° C.

In still another embodiment, the solid dispersion obtained in the present inventions under the conditions selected at a temperature of 20-60° C., preferably 25-30° C. and at a relative humidity of 50-70%, preferably 55-65%.

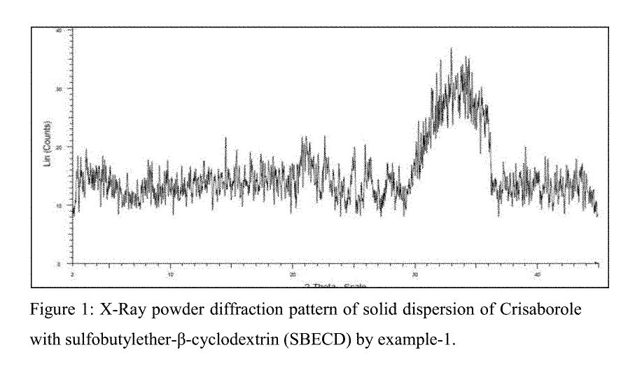

Yet in another embodiment, the solid dispersions obtained by any of the above methods were analysed using X-ray powder diffractometer and characterized by the X-ray powder diffraction graphs as shown in FIG. 1 to FIG. 72 with the below mentioned operating parameters.

Operation Parameters:

Name of instrument: (D8 Advance, Bruker Corporation, Germany)

wavelength: λ=1.54056 A) using Cu-Kn radiation

Detector: Scintillation counter

Voltage (kV), Current (mA): 40,35

Anode: Copper

Scan Type, Scan Mode: Locked, Coupled

Angular Range (°2∂): 4 to 6

Step size (°): 0.0050

Time per Step (Seconds): 6

The following examples further illustrate the present invention, but should not be construed in anyway, as to limit its scope.

### EXAMPLES

### Example-1: Preparation of Solid Dispersion of Crisaborole with Sulfobutylether-β-Cyclodextrin (SBECD) (1:1)

1.0 g of Crisaborole was dissolved in 5 volumes of isopropyl alcohol and stirred for 5-10 minutes at 25-30° C. To this, 1.0 g of sulfobutylether-β-cyclodextrin (SBECD) and 7 volumes of water were added. Then the reaction mixture was heated for 30 minutes at 60-65° C. and cooled to 25-30° C. The obtained reaction mixture was lyophilized to obtain solid dispersion of Crisaborole with sulfobutylether-β-cyclodextrin (SBECD). Yield: 1.9 g; XRD: FIG. 1.

### Example-2: Preparation of Solid Dispersion of Eliglustat Hemitartrate with Sulfobutylether-β-Cyclodextrin (SBECD) (1:1)

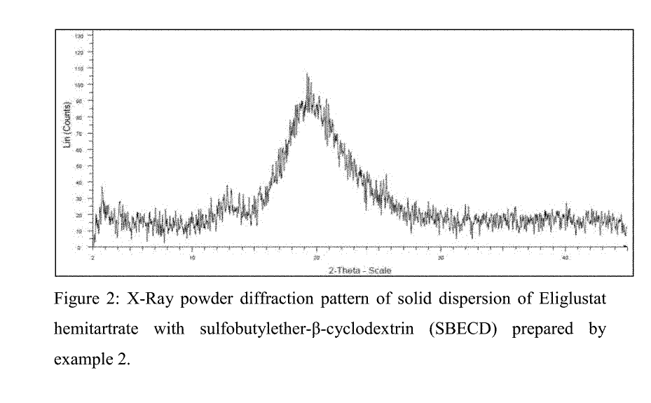

1.0 g of Eliglustat hemitartrate was dissolved in 10 volumes of water and stirred at 25-30° C. to get a clear solution. 1.0 g of sulfobutylether-β-cyclodextrin (SBECD) was added to the solution and stirred for 15-20 minutes at 25-30° C. The reaction mixture was lyophilized to obtain solid dispersion of Eliglustat hemitartrate with sulfobutylether-β-cyclodextrin (SBECD). The product so obtained was stored under nitrogen atmosphere. Yield: 1.98 g; XRD: FIG. 2.

### Example-3: Preparation of Solid Dispersion of Eliglustat Hemitartrate with Sulfobutylether-β-Cyclodextrin (SBECD) (1:2)

1.0 g of Eliglustat hemitartrate was dissolved in 10 volumes of water and stirred at 25-30° C. to get a clear solution. 2.0 g of sulfobutylether-β-cyclodextrin (SBECD) was then added to the clear solution and stirred for 20-30 minutes at 25-30° C. The reaction mixture was lyophilized to obtain solid dispersion of Eliglustat hemitartrate with sulfobutylether-β-cyclodextrin (SBECD). The product so obtained was stored under nitrogen atmosphere. Yield: 2.78 g;

### Example-4: Preparation of Solid Dispersion of Eliglustat Hemitartrate with Sulfobutylether-β-Cyclodextrin (SBECD) (1:0.6)

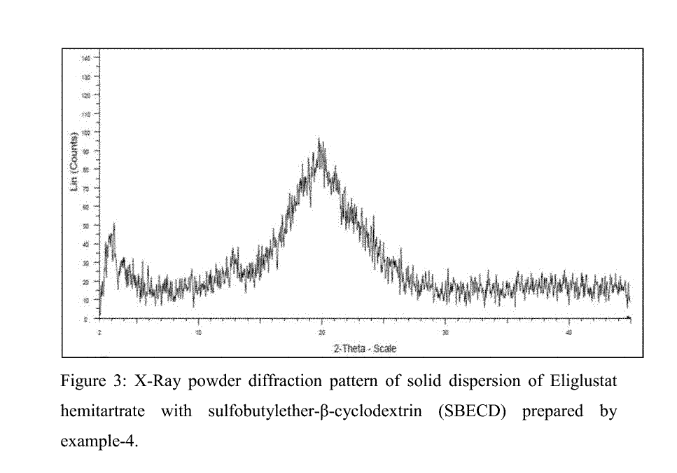

1.0 g of Eliglustat hemitartrate was dissolved in 10 volumes of water and stirred at 25-30° C. to get a clear solution. 0.6 g of sulfobutylether-β-cyclodextrin (SBECD) was then added to the clear solution and stirred for at 25-30° C. The reaction mixture was lyophilized to obtain solid dispersion of Eliglustat hemitartrate with sulfobutylether-β-cyclodextrin (SBECD). The product so obtained was stored under nitrogen atmosphere. Yield: 1.54 g; XRD: FIG. 3.

### Example-5: Preparation of Solid Dispersion of Eliglustat Hemitartrate with Sulfobutylether-β-Cyclodextrin (SBECD) (1:0.3)

1.0 g Eliglustat hemitartrate was dissolved in 10 volumes of water and stirred at 25-30° C. to get a clear solution. 0.3 g of sulfobutylether-β-cyclodextrin (SBECD) was then added to the clear solution and stirred for at 25-30° C. The reaction mass was lyophilized to obtain solid dispersion of Eliglustat hemitartrate (I) with sulfobutylether-β-cyclodextrin (SBECD). The product so obtained was stored under nitrogen atmosphere. Yield: 1.27 g;

### Example-6: Preparation of Solid Dispersion of Cariprazine Hydrochloride with Sulfobutylether-β-Cyclodextrin (SBECD) (1:6)

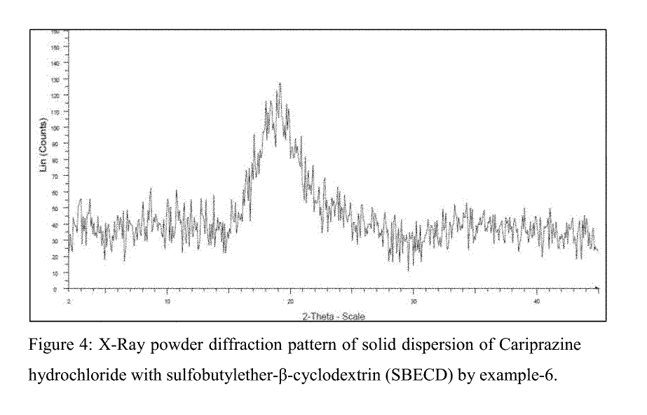

1.0 g of Cariprazine hydrochloride was dissolved in 120 mL of dimethyl sulfoxide. 6.0 g of sulfobutylether-β-cyclodextrin (SBECD) was then added to the reaction mixture, stirred for 15-30 minutes at 50-60° C. to form a clear solution. The clear solution was micron filtered, and the filtrate was lyophilized to obtain solid dispersion of Cariprazine hydrochloride with sulfobutylether-β-cyclodextrin (SBECD). The product so obtained was stored under nitrogen atmosphere. Yield: 98.2%; XRD: FIG. 4.

### Example-7: Preparation of Solid Dispersion of Eliglustat Hemitartrate with Hydroxypropyl Beta Cyclodextrin (HPBCD) (1:1)

1.0 g of Eliglustat hemitartrate was dissolved in 10 volumes of water and stirred at 25-30° C. to get a clear solution. 1.0 g of hydroxypropyl beta cyclodextrin (HPBCD) was added to the clear solution and stirred at 25-30° C. The reaction mixture was lyophilized to obtain solid dispersion of Eliglustat hemitartrate with hydroxypropyl beta cyclodextrin (HPBCD). Yield: 1.85 g;

### Example-8: Preparation of Solid Dispersion of Eliglustat Hemitartrate with Hydroxypropyl Beta Cyclodextrin (HPBCD) (1:2)

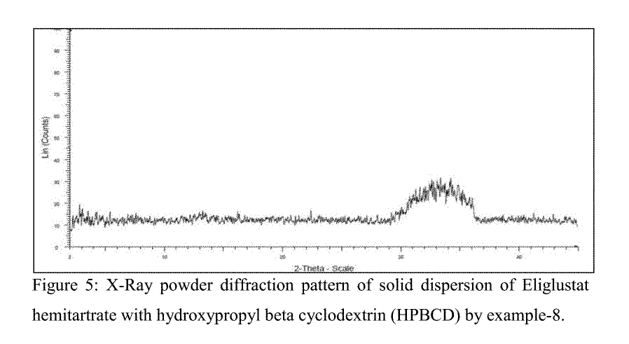

1.0 g of Eliglustat hemitartrate was dissolved in 10 volumes of water and stirred at 25-30° C. to get a clear solution. 2.0 g of hydroxypropyl beta cyclodextrin (HPBCD) was added to the clear solution and stirred at 25-30° C. The reaction mixture so obtained was lyophilized to yield solid dispersion of Eliglustat hemitartrate with hydroxypropyl beta cyclodextrin (HPBCD). Yield: 2.89 g; XRD: FIG. 5.

### Example-9: Preparation of Solid Dispersion of Eliglustat Hemitartrate with Hydroxypropyl Beta Cyclodextrin (HPBCD) (1:0.6)

1.0 g of Eliglustat hemitartrate was dissolved in 10 volumes of water and stirred at 25-30° C. to get a clear solution. 0.6 g of hydroxypropyl beta cyclodextrin (HPBCD) was added to the clear solution and stirred at 25-30° C. The reaction mixture so obtained was lyophilized to yield solid dispersions of Eliglustat hemitartrate with hydroxypropyl beta cyclodextrin (HPBCD). Yield: 1.55 g;

### Example-10: Preparation of Solid Dispersion of Eliglustat Hemitartrate with Hydroxypropyl Beta Cyclodextrin (HPBCD) (1:0.3)

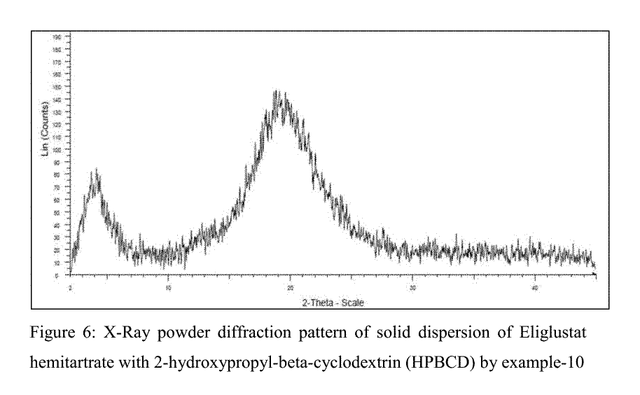

1.0 g of Eliglustat hemitartrate was dissolved in 10 volumes of water and stirred at 25-30° C. to get a clear solution. 0.3 g of hydroxypropyl beta cyclodextrin (HPBCD) was added to the clear solution and stirred at 25-30° C. The reaction mixture was lyophilized to obtain solid dispersion of Eliglustat hemitartrate with hydroxypropyl beta cyclodextrin (HPBCD). Yield: 1.25 g; XRD: FIG. 6.

### Example-11: Preparation of Solid Dispersion of Eliglustat Hemitartrate with γ-Cyclodextrin (1:1)

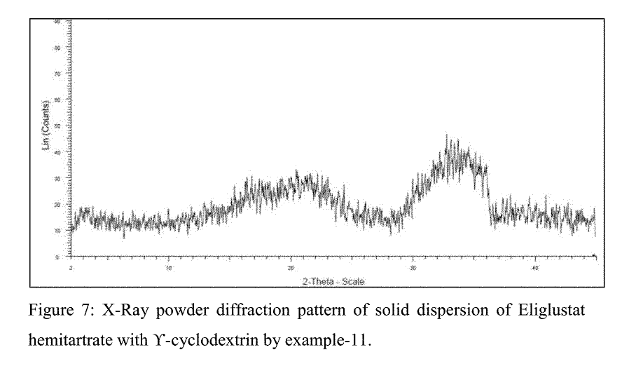

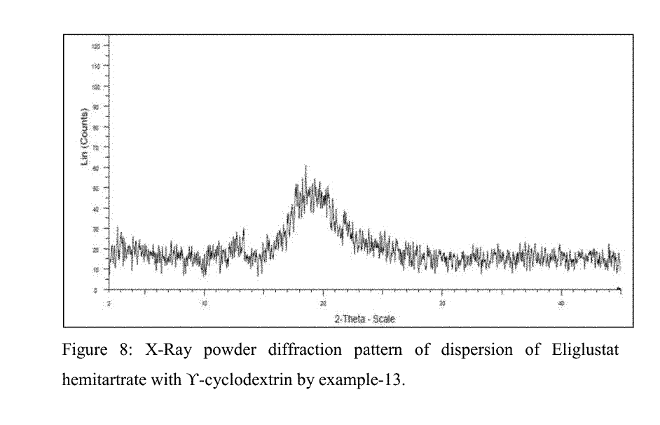

1.0 g of Eliglustat hemitartrate was dissolved in 10 volumes of water and stirred at 25-30° C. to get a clear solution. 1.0 g of Υ-cyclodextrin was added to the clear solution and stirred at 25-30° C. The reaction mixture so obtained was lyophilized to obtain solid dispersion of Eliglustat hemitartrate with Υ-cyclodextrin. Yield: 1.91 g; XRD: FIG. 7.

### Example-12: Preparation of Solid Dispersion of Eliglustat Hemitartrate with γ-Cyclodextrin (1:2)

1.0 g of Eliglustat hemitartrate was dissolved in 10 volumes of water and stirred at 25-30° C. to get a clear solution. 2.0 g of Υ-cyclodextrin was then added to the clear solution and stirred at 25-30° C. The reaction mixture so obtained was lyophilized to obtain solid dispersion of Eliglustat hemitartrate with Υ-cyclodextrin. Yield: 2.96 g;

### Example-13: Preparation of Solid Dispersion of Eliglustat Hemitartrate with γ-Cyclodextrin (1:0.6)

1.0 g of Eliglustat hemitartrate was dissolved in 10 volumes of water and stirred at 25-30° C. to get a clear solution. 0.6 g of Υ-cyclodextrin was then added to the clear solution and stirred at 25-30° C. The reaction mixture so obtained was lyophilized to obtain solid dispersion of Eliglustat hemitartrate with Υ-cyclodextrin. Yield: 1.57 g; XRD: FIG. 8.

### Example-14: Preparation of Solid Dispersion of Eliglustat Hemitartrate with γ-Cyclodextrin (1:0.3)

1.0 g of Eliglustat hemitartrate was dissolved in 10 volumes of water and stirred at 25-30° C. to get a clear solution. 0.3 g of Υ-cyclodextrin was then added to the clear solution and stirred at 25-30° C. The reaction mixture so obtained was lyophilized to obtain solid dispersion of Eliglustat hemitartrate (I) with Υ-cyclodextrin. Yield: 1.28 g;

### Example-15: Preparation Solid Dispersion of Tipiracil Hydrochloride (1) with Polyvinylpyrrolidone (PVP) (1:1)

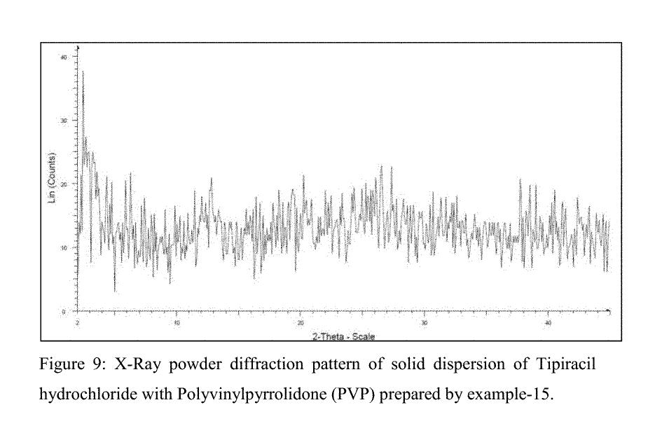

1.0 g of Tipiracil hydrochloride was dissolved in 15 volumes of water and stirred at 25-30° C. to form a clear solution. 1.0 g of poly vinyl povidone (PVP) was then added to the clear solution and stirred for 30 minutes at 25-30° C. The reaction mixture so obtained was lyophilized to acquire solid dispersion of Tipiracil hydrochloride with poly vinyl povidone (PVP). The product so obtained was stored under nitrogen atmosphere with desiccants. Yield: 1.8 g; XRD: FIG. 9.

### Example-16: Preparation of Solid Dispersion of Tipiracil Hydrochloride with Polyvinylpyrrolidone (PVP) (1:2)

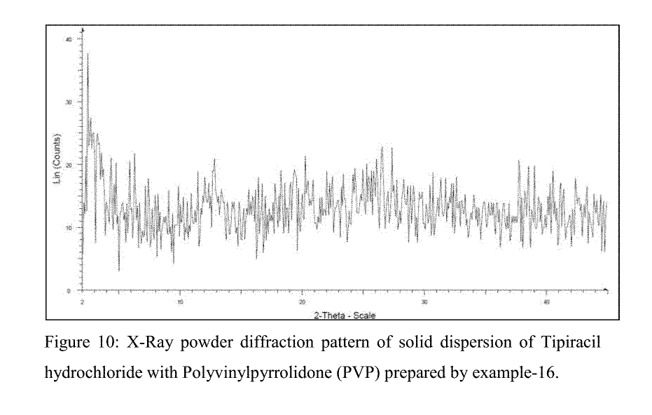

5.0 g of Tipiracil hydrochloride was dissolved in 50 volumes of water and stirred at 25-30° C. to get a clear solution. 10.0 g of poly vinyl povidone (PVP) was then added to the clear solution and stirred for 30 minutes at 25-30° C. The reaction mixture so obtained was lyophilized to yield solid dispersion of Tipiracil hydrochloride with poly vinyl povidone (PVP). Yield: 12.0 g; XRD: FIG. 10.

### Example-17: Preparation of Solid Dispersion of Crisaborole with Polyvinylpyrrolidone (PVP) (1:1)

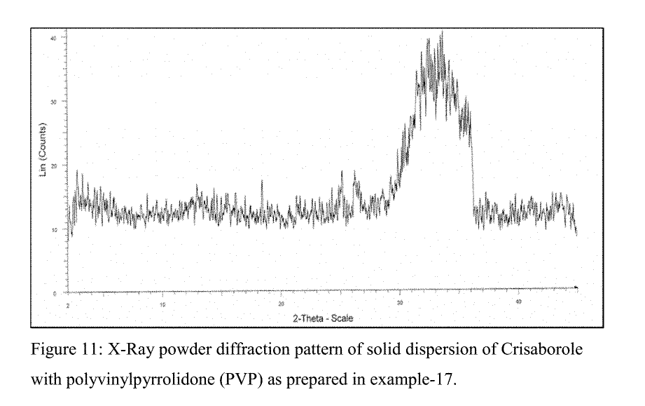

1.0 g of Crisaborole was dissolved in 5 volumes of isopropyl alcohol and stirred for 5-10 minutes at 25-30° C. 1.0 g of poly vinyl pyrrolidone (PVP) and 7 volumes of water added and heated for 30 minutes at 60-65° C. Then the reaction mixture was cooled to 25-30° C. and lyophilized to obtain solid dispersion of Crisaborole with of polyvinylpyrrolidone (PVP). Yield: 1.8 g; XRD: FIG. 11.

### Example-18: Preparation of Solid Dispersion of Tipiracil Hydrochloride with Lactose (1:1)

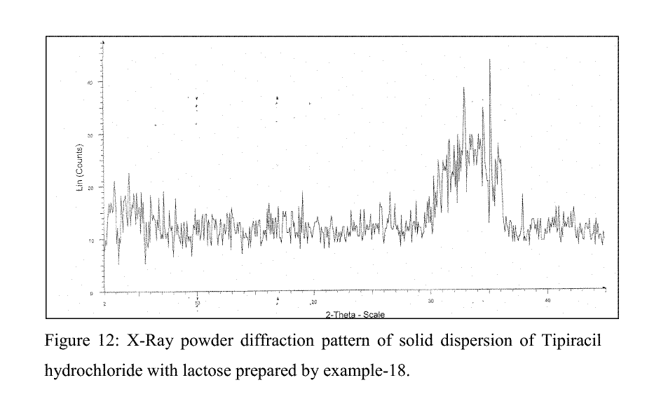

1.0 g of Tipiracil hydrochloride was dissolved in 5 volumes of isopropyl alcohol and stirred at 25-30° C. to get a clear solution. 1.0 g of lactose was added to the clear solution and stirred for 30 minutes at 25-30° C. The reaction mass so obtained was lyophilized to yield solid dispersion of Tipiracil hydrochloride with lactose. Yield: 1.2 g; XRD: FIG. 12

### Example-19: Preparation of Solid Dispersion of Tipiracil Hydrochloride with Hydroxy Propyl Methyl Cellulose (HPMC) (1:3)

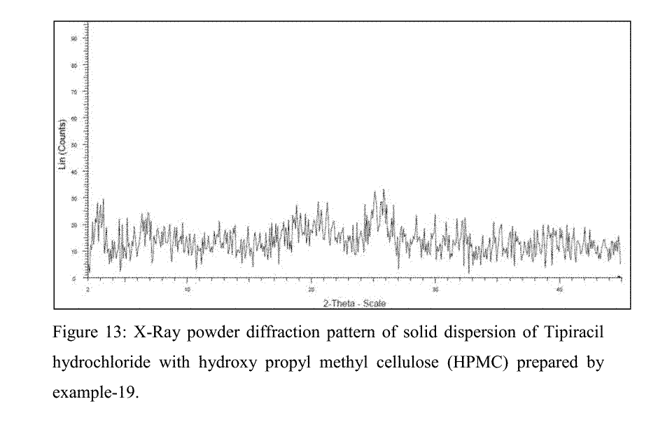

1.0 g of Tipiracil hydrochloride was dissolved in 15 volumes of water and stirred at 25-30° C. to get a clear solution. 3.0 g of hydroxy propyl methyl cellulose was added and stirred for 30 minutes at 25-30° C. The reaction mixture so obtained was lyophilized to acquire solid dispersion of Tipiracil hydrochloride with hydroxy propyl methyl cellulose. Yield: 3.7 g; XRD: FIG. 13

### Example-20: Preparation of Solid Dispersion of Tipiracil Hydrochloride with Hydroxy Propyl Methyl Cellulose (HPMC) (1:1)

1.0 g of Tipiracil hydrochloride was dissolved in 15 volumes of water and stirred at 25-30° C. to get a clear solution. 1.0 g of hydroxy propyl methyl cellulose was then added to the clear solution and stirred for 30 minutes at 25-30° C. The reaction mass so obtained was lyophilized to acquire solid dispersion of Tipiracil hydrochloride with hydroxy propyl methyl cellulose. The product so obtained was stored under nitrogen atmosphere. Yield: 1.8 g;

### Example-21: Alternative Preparation of Solid Dispersion of Tipiracil Hydrochloride with Hydroxy Propyl Methyl Cellulose (HPMC) (1:2)

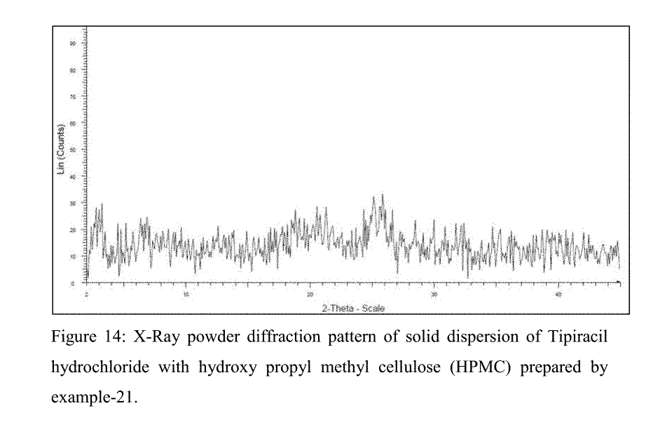

1.0 g of Tipiracil hydrochloride was dissolved in 15 volumes of water and stirred at 25-30° C. to get a clear solution. 2.0 g of hydroxy propyl methyl cellulose was added to the clear solution and stirred for 30 minutes. The reaction mixture so obtained was lyophilized to acquire solid dispersion of Tipiracil hydrochloride with hydroxy propyl methyl cellulose. Yield: 2.8 g; XRD: FIG. 14.

### Example-22: Preparation of Solid Dispersion of Tipiracil Hydrochloride with Hydroxy Propyl Methyl Cellulose (HPMC) (1:3)

10.0 g of Tipiracil hydrochloride was dissolved in 150 volumes of water and stirred at 25-30° C. to form a clear solution. 30.0 g of hydroxy propyl methyl cellulose was added to the clear solution and stirred for 30 minutes at 25-30° C. The reaction mass so obtained was lyophilized to acquire solid dispersion of Tipiracil hydrochloride with hydroxy propyl methyl cellulose. Yield: 39 g;

### Example-23: Preparation of Solid Dispersion of Tipiracil Hydrochloride with Hydroxy Propyl Methyl Cellulose (HPMC) (1:2)

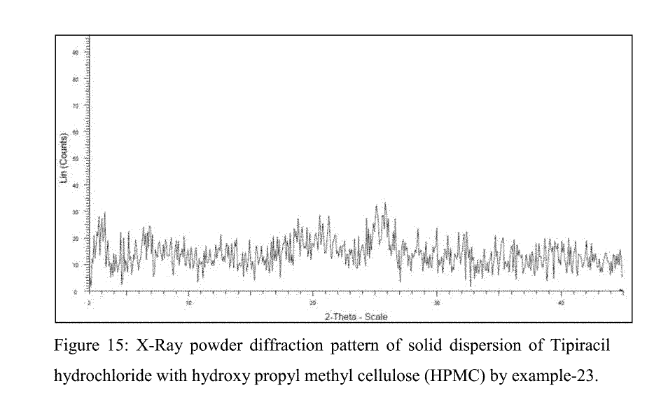

5.0 g of Tipiracil hydrochloride was dissolved in 50 volumes of water and stirred at 25-30° C. to get a clear solution. 10.0 g of hydroxy propyl methyl cellulose was then added to the clear solution and stirred for 30 minutes at 25-30° C. The reaction mixture so obtained was lyophilized to yield solid dispersion of Tipiracil hydrochloride with hydroxy propyl methyl cellulose. Yield: 15 g; XRD: FIG. 15.

### Example-24: Preparation of Solid Dispersion of Tipiracil Hydrochloride with Hydroxy Propyl Methyl Cellulose (HPMC)(1:3)

1.0 g of Tipiracil hydrochloride was dissolved in 15 volumes of water and stirred at 25-30° C. to get a clear solution. 3.0 g of hydroxy propyl methyl cellulose was added to the clear solution and stirred for 30 minutes at 25-30° C. The reaction mass so obtained was lyophilized to yield solid dispersion of Tipiracil hydrochloride with hydroxy propyl methyl cellulose. Yield: 3.7 g;

### Example-25: Preparation of Solid Dispersion of Crisaborole with Hydroxypropyl Methyl Cellulose (HPMC) (1:1)

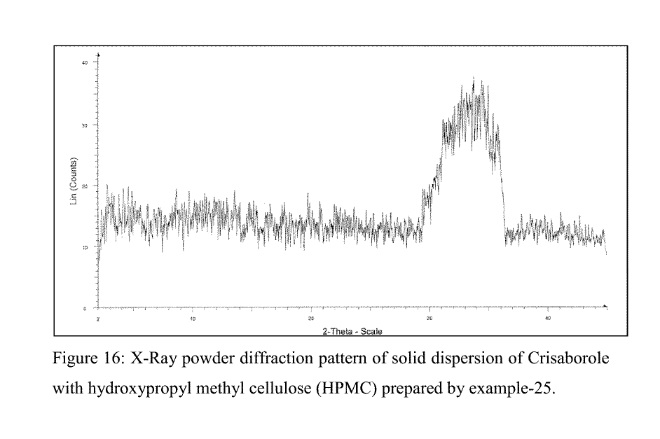

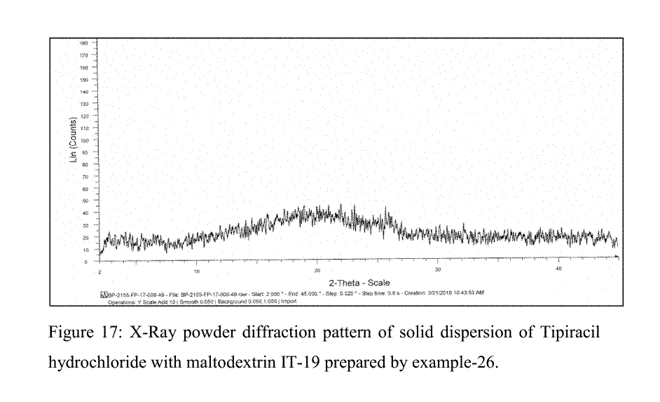

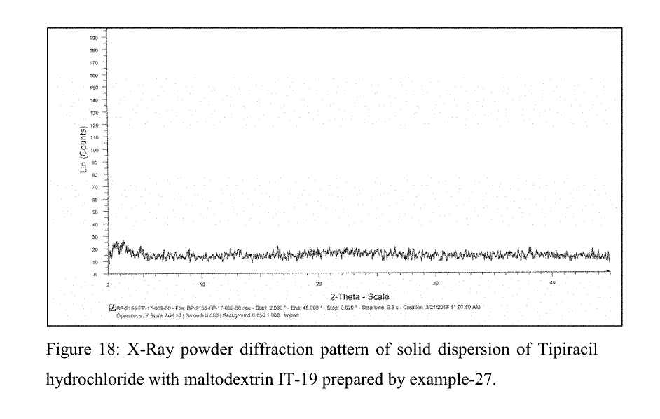

1.0 g of Crisaborole was dissolved in 5 volumes of isopropyl alcohol and stirred for 5-10 minutes to form a clear solution at 25-30° C. 1.0 g of hydroxypropyl methyl cellulose (HPMC) and 10 volumes of water were added. The reaction mixture was heated for 30 minutes at 60-65° C. Then the reaction mixture was cooled to 25-30° C. and lyophilized to obtain solid dispersion of Crisaborole with hydroxypropyl methyl cellulose. Yield: 1.8 g; XRD: FIG. 16.

### Example-26: Preparation of Solid Dispersion of Tipiracil Hydrochloride with Maltodextrin IT-19 (1:2.5)

1.0 g of Tipiracil hydrochloride was dissolved in 10 volumes of water and stirred at 25-30° C. to get a clear solution. 2.5 g of maltodextrin IT-19 was added to the clear solution and stirred for 30 minutes at 25-30° C. The reaction mass so obtained was lyophilized to yield solid dispersion of Tipiracil hydrochloride with maltodextrin IT-19. Yield: 3.2 g; XRD: FIG. 17.

### Example-27: Preparation of Solid Dispersion of Tipiracil Hydrochloride with Maltodextrin IT-19 (1:5)

1.0 g of Tipiracil hydrochloride was dissolved in 10 volumes of water and stirred at 25-30° C. to get a clear solution. 5.0 g of maltodextrin IT-19 was added to the clear solution and stirred for 30 minutes at 25-30° C. The reaction mass so obtained was lyophilized to yield solid dispersion of Tipiracil hydrochloride with maltodextrin IT-19. Yield: 5.8 g; XRD: FIG. 18

### Example-28: Preparation of Solid Dispersion of Ribociclib Succinate with Sulfobutylether-β-Cyclodextrin (SBECD) (1:1)

1.0 g of Ribociclib succinate (1) and 1.0 g of sulfobutylether-β-cyclodextrin (SBECD) were suspended in a mixture of 20 mL methanol and 20 mL dichloromethane and stirred for 10 minutes at 25-30° C. The reaction mixture was then heated to 50-65° C. and stirred for 15 minutes to form a clear solution. The solvent was evaporated by using rotary evaporator under vacuum at 50-65° C. to obtain solid dispersion of Ribociclib succinate with sulfobutylether-β-cyclodextrin (SBECD). Yield: 69%; XRD: FIG. 19.

### Example-29: Preparation of Solid Dispersion of Ribociclib Succinate with Sulfobutylether-β-Cyclodextrin (SBECD)(1:0.5)

1.0 g of Ribociclib succinate and 0.5 g of sulfobutylether-β-cyclodextrin (SBECD) were suspended in a mixture of 20 mL methanol and 30 mL dichloromethane and stirred for 10 minutes at 25-30° C. The reaction mixture was then heated to 50-65° C. and stirred for 15 minutes to form a clear solution. The solvent was evaporated by using rotary evaporator under vacuum at 50-65° C. to obtain solid dispersion of Ribociclib succinate (1) with sulfobutylether-β-cyclodextrin (SBECD). Yield: 70%; XRD: FIG. 20

### Example-30: Preparation of Solid Dispersion of Ribociclib Succinate with Sulfobutylether-β-Cyclodextrin (SBECD) (1:0.75)

1.0 g of Ribociclib succinate and 0.75 g of sulfobutylether-β-cyclodextrin (SBECD) were suspended in a mixture of 20 mL methanol and 30 mL dichloromethane and stirred for 10 minutes at 25-30° C. The reaction mixture was then heated to 50-65° C. and stirred for 15 minutes to form a clear solution. The solvent was evaporated by using rotary evaporator under vacuum at 50-65° C. to obtain solid dispersion of Ribociclib succinate with sulfobutylether-β-cyclodextrin (SBECD). Yield: 72%;

### Example-31: Preparation of Solid Dispersion of Eluxadoline with Sulfobutylether-β-Cyclodextrin (SBECD) (1:1)

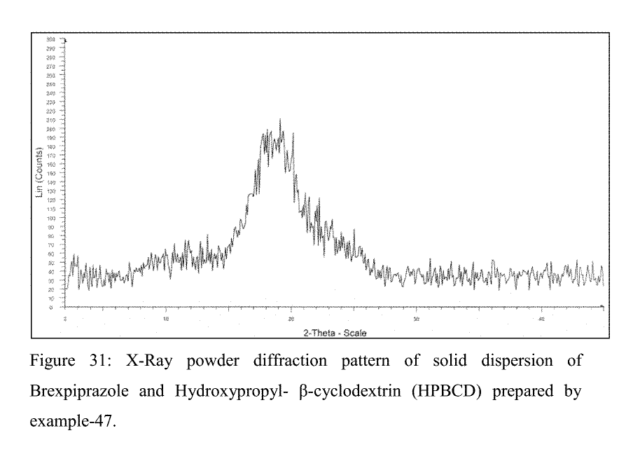

1.0 g of Eluxadoline and 1.0 g of sulfobutylether-β-cyclodextrin (SBECD) were suspended in 100 mL methanol and stirred for 15-20 minutes at 25-30° C. The reaction mixture was heated to 50-65° C. and stirred for 15-20 minutes to form a clear solution. The solvent of the clear solution was then removed by distillation to obtain solid dispersion of Eluxadoline (1) with sulfobutylether-β-cyclodextrin (SBECD). Yield: 84%; XRD: FIG. 31.

### Example-32: Preparation of Solid Dispersion of Eluxadoline with Sulfobutylether-β-Cyclodextrin (SBECD) (1:0.5)

1.0 g of Eluxadoline and 0.5 g of sulfobutylether-β-cyclodextrin (SBECD) were suspended in 100 mL methanol and stirred for 15-20 minutes at 25-30° C. The reaction mixture was heated to 50-65° C. and stirred for 15-20 minutes to form a clear solution. The solvent of the clear solution was then removed by distillation to obtain solid dispersion of Eluxadoline (1) with sulfobutylether-β-cyclodextrin (SBECD). Yield: 86%;

### Example-33: Preparation of Solid Dispersion of Ribociclib Succinate with Sulfobutylether-β-Cyclodextrin (SBECD) (1:1)

1.0 g of Ribociclib succinate and 1.0 g of sulfobutylether-β-cyclodextrin (SBECD) were suspended in a mixture of 20 mL methanol and 20 mL dichloromethane and stirred for 10 minutes at 25-30° C. The reaction mixture was then heated to 50-65° C. and stirred for 15 minutes to form a clear solution. The solvent of the clear solution was distilled off using rotary evaporator under vacuum at 50-65° C. to obtain solid dispersion of Ribociclib succinate with sulfobutylether-β-cyclodextrin (SBECD). Yield: 69%; XRD: FIG. 22.

### Example-34: Preparation of Solid Dispersion of Ribociclib Succinate with Sulfobutylether-β-Cyclodextrin (SBECD) (1:0.5)

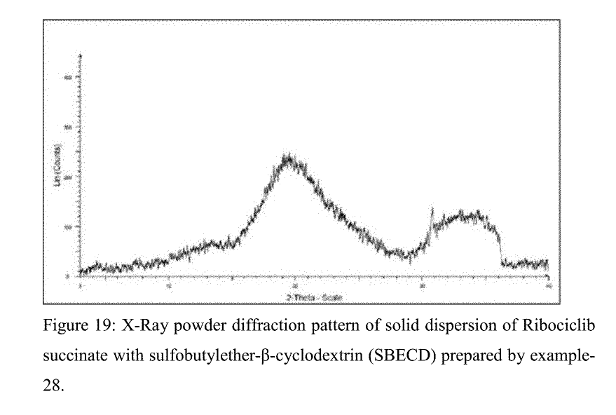

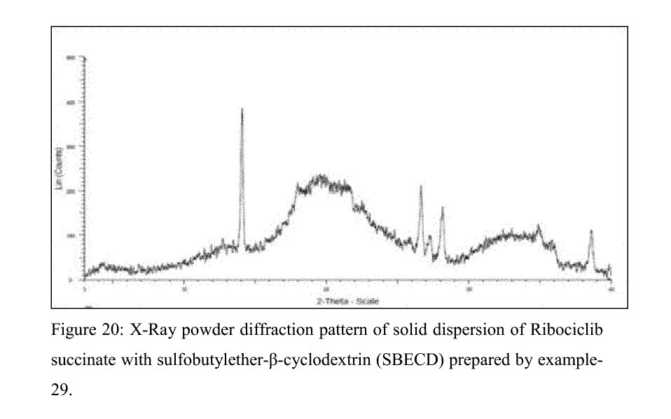

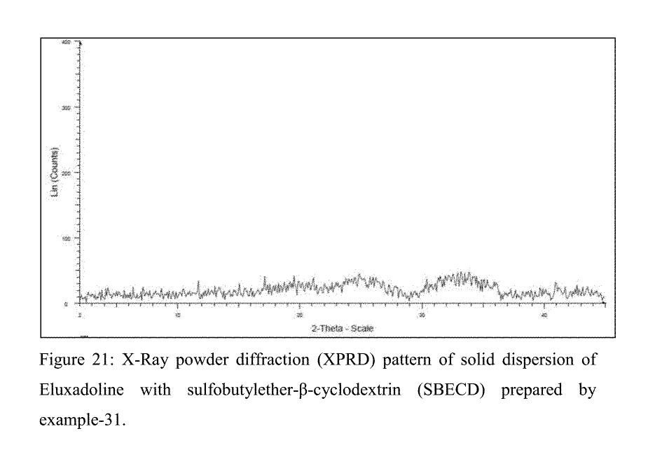

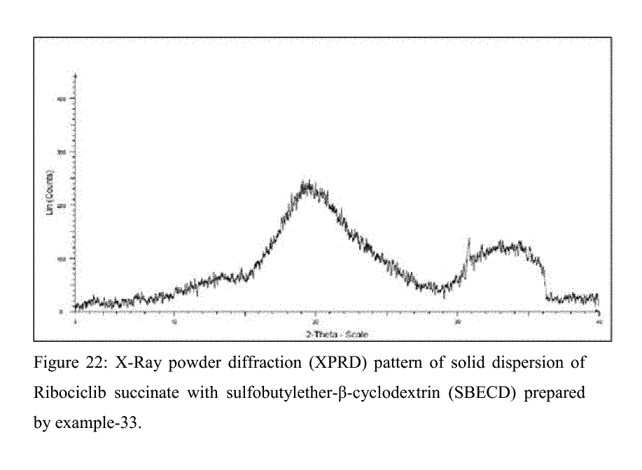

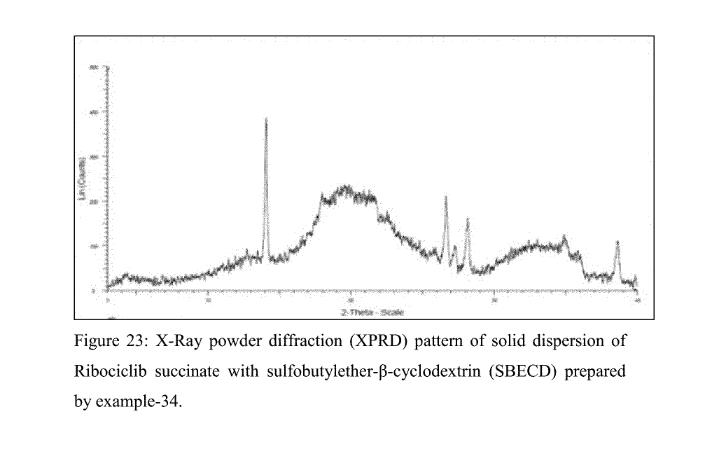

1.0 g of Ribociclib succinate and 0.5 g of sulfobutylether-β-cyclodextrin (SBECD) were suspended in a mixture of 20 mL methanol and 30 mL dichloromethane and stirred for 10 minutes at 25-30° C. The reaction mixture was then heated to 50-65° C. and stirred for 15 minutes to form a clear solution. The solvent of the clear solution was distilled off using rotary evaporator under vacuum at 50-65° C. to obtain solid dispersion of Ribociclib succinate with sulfobutylether-β-cyclodextrin (SBECD). Yield: 70%; XRD: FIG. 23

### Example-35: Preparation of Solid Dispersion of Ribociclib Succinate with Sulfobutylether-β-Cyclodextrin (SBECD) (1:0.75)

1.0 g of Ribociclib succinate and 0.75 g of sulfobutylether-β-cyclodextrin (SBECD) were suspended in a mixture of 20 mL methanol and 30 mL dichloromethane and stirred for 10 minutes at 25-30° C. The reaction mixture was then heated to 50-65° C. and stirred for 15 minutes to form a clear solution. The solvent of the clear solution was distilled off using rotary evaporator under vacuum at 50-65° C. to obtain solid dispersion of Ribociclib succinate with sulfobutylether-β-cyclodextrin (SBECD). Yield: 72%;

### Example-36: Preparation of Solid Dispersion of Cariprazine Hydrochloride with Sulfobutylether-β-Cyclodextrin (SBECD) (1:6)

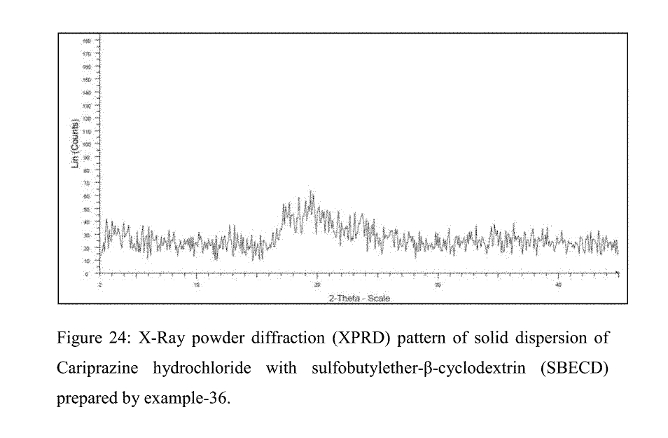

1.0 g of Cariprazine hydrochloride was dissolved in 200 mL methanol and 6.0 g of sulfobutylether-β-cyclodextrin (SBECD) was added at 25-30° C. The reaction mixture was heated to 55-60° C. and stirred for 15-30 minutes to form a clear solution and passed through micron filter. The filtrate was evaporated by using rotary evaporator below 65° C. under high vacuum and dried to obtain solid dispersion of Cariprazine hydrochloride with sulfobutylether-β-cyclodextrin (SBECD). Yield: 94.2%; XRD: FIG. 24.

### Example-37: Preparation of Solid Dispersion of Rucaparib Camsylate with Sulfobutylether-β-Cyclodextrin (SBECD) (1:0.5)

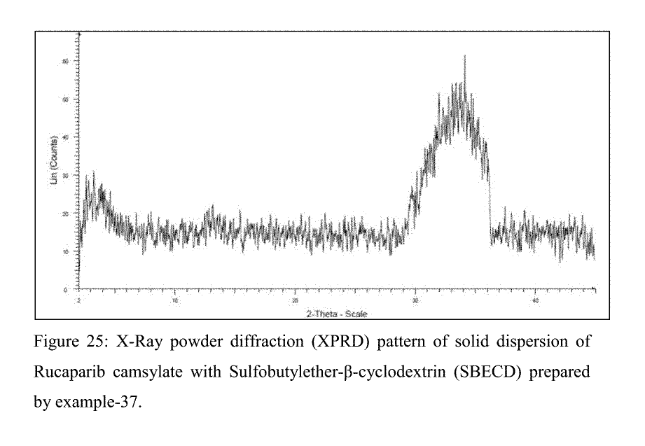

2.0 g of Rucaparib camsylate was suspended in 40 mL of methanol and 1.0 g of sulfobutylether-β-cyclodextrin (SBECD) was added at 25-30° C. The reaction mixture was then heated at 55-60° C. and 4 mL of water was added to it. The reaction mixture was stirred for 15-30 minutes at 55-60° C. to form a clear solution and filtered. The filtrate was evaporated by using rotary evaporator under vacuum and the residue dried under vacuum below 60° C. to obtain solid dispersion of Rucaparib camsylate with sulfobutylether-β-cyclodextrin (SBECD). Yield: 93%; XRD: FIG. 25.

### Examples-38: Preparation of Solid Dispersion of Brexpiprazole and Sulfobutylether-β-Cyclodextrin (SBECD) (1:3)

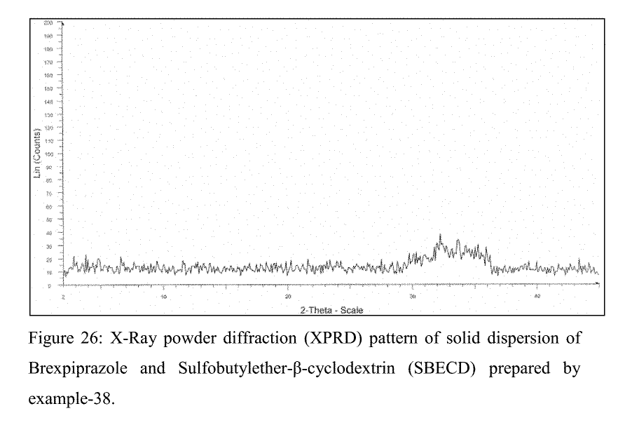

1.0 g of Brexpiprazole of formula was suspended in 20 mL of dichloromethane, a solution of 3.0 g of sulfobutylether-β-cyclodextrin (SBECD) in 160 mL methanol was added at 25-35° C. The reaction mixture was heated to about 60-65° C. for 30 minutes and filtered. The clear solution was taken into a Buchi flask and the solvent evaporated completely and dried at below 65° C. to get the solid dispersion of Brexpiprazole and Sulfobutylether-β-cyclodextrin (SBECD). Yield: 96%; XRPD: FIG. 26.

### Example-39: Preparation of Solid Dispersion of Eluxadoline with Hydroxypropyl Beta Cyclodextrin (HPBCD) (1:0.5)

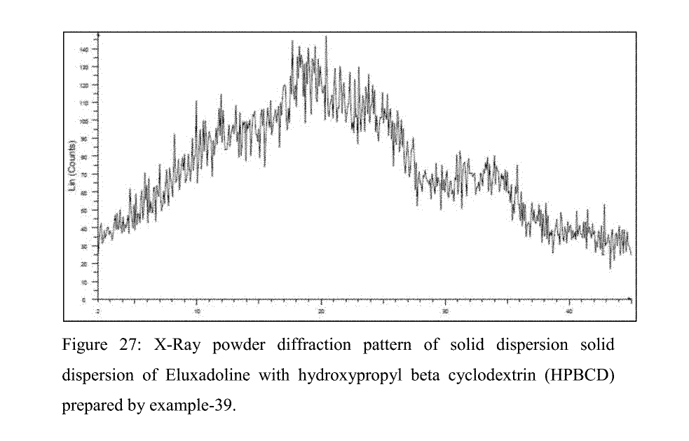

1.0 g of Eluxadoline and 0.5 g of hydroxypropyl beta cyclodextrin (HPBCD) were suspended in 100 mL methanol at 25-30° C. The reaction mixture was then heated to 50-65° C. and stirred for 30 minutes to form a clear solution. The solvent of the clear solution was removed by distillation to obtain solid dispersion of Eluxadoline with hydroxypropyl beta cyclodextrin (HPBCD). Yield: 85%; XRD: FIG. 27.

### Example-40: Preparation of Solid Dispersion of Eluxadoline with Hydroxypropyl Beta Cyclodextrin (HPBCD) (1:1)

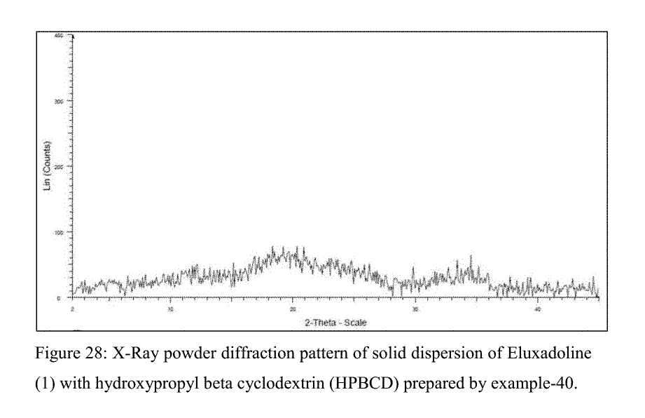

1.0 g of Eluxadoline and 1.0 g of hydroxypropyl beta cyclodextrin (HPBCD) were suspended in 100 mL methanol at 25-30° C. The reaction mixture was then heated to 50-65° C. and stirred for 30 minutes to form a clear solution. The solvent was evaporated by using rotary evaporator to obtain solid dispersion of Eluxadoline with hydroxypropyl beta cyclodextrin (HPBCD). Yield: 83%; XRD: FIG. 28.

### Example-41: Preparation of Solid Dispersion of Eluxadoline with Hydroxypropyl Beta Cyclodextrin (HPBCD) (1:1.5)

1.0 g of Eluxadoline and 1.5 g of hydroxypropyl beta cyclodextrin (HPBCD) were suspended in 120 mL methanol at 25-30° C. and stirred for 15 minutes. The reaction mixture was then heated to 50-65° C. for 30 minutes to form a clear solution. The solvent was then removed by distillation to obtain solid dispersion of Eluxadoline (1) with hydroxypropyl beta cyclodextrin (HPBCD). Yield: 82%;

### Example-42: Preparation of Solid Dispersion of Ribociclib Succinate with Hydroxypropyl Beta Cyclodextrin (HPBCD)(1:1.5)

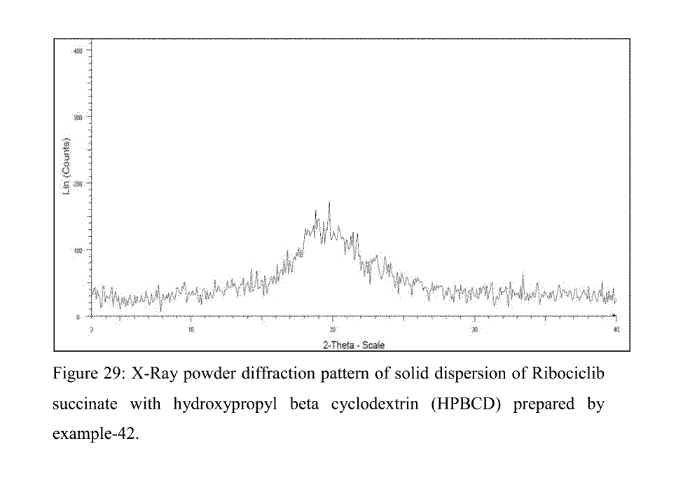

1.0 g of Ribociclib succinate and 1.5 g of hydroxypropyl beta cyclodextrin (HPBCD) were suspended in a mixture of 10 mL methanol and 10 mL dichloromethane and stirred for 15 minutes at 25-30° C. The reaction mixture was then heated to 50-65° C. and stirred for 15 minutes to form a clear solution. The solvent was evaporated by using rotary evaporator under vacuum at 50-65° C. to obtain solid dispersion of Ribociclib succinate with hydroxypropyl beta cyclodextrin (HPBCD). Yield: 76%; XRD: FIG. 29.

### Example-43: Preparation of Solid Dispersion of Eluxadoline with Hydroxypropyl Beta Cyclodextrin (HPBCD) (1:1)

1.0 g of Eluxadoline and 1.0 g of hydroxypropyl beta cyclodextrin (HPBCD) were suspended in 150 mL methanol at 25-30° C. The reaction mixture was then heated to 50-65° C. and stirred for 30 minutes to form a clear solution. The solvent was removed by distillation to obtain solid dispersion of Eluxadoline with hydroxypropyl beta cyclodextrin (HPBCD). Yield: 83%;

### Example-44: Preparation of Solid Dispersion of Eluxadoline with Hydroxypropyl Beta Cyclodextrin (HPBCD) (1:1.5)

1.0 g of Eluxadoline and 1.5 g of hydroxypropyl beta cyclodextrin (HPBCD) were suspended in 150 mL methanol at 25-30° C. and stirred for 15 minutes. The reaction mixture was then heated to 50-65° C. for 30 minutes to form a clear solution. The solvent of the clear solution was removed completely by distillation to obtain solid dispersion of Eluxadoline with hydroxypropyl beta cyclodextrin (HPBCD). Yield: 82%;

### Example-45: Preparation of Solid Dispersion of Ribociclib Succinate with Hydroxypropyl Beta Cyclodextrin (HPBCD) (1:1.5)

1.0 g of Ribociclib succinate and 1.5 g of hydroxypropyl beta cyclodextrin (HPBCD) were suspended in a mixture of 20 mL methanol and 20 mL dichloromethane and stirred for 15 minutes at 25-30° C. The reaction mixture was then heated to 50-65° C. and stirred for 15 minutes to form a clear solution. The solvent was distilled off using rotary evaporator under vacuum at 50-65° C. to obtain solid dispersion of Ribociclib succinate (1) with hydroxypropyl beta cyclodextrin (HPBCD). Yield: 76%;

### Example-46: Preparation of Solid Dispersion of Cariprazine Hydrochloride with Hydroxypropyl Beta Cyclodextrin (HPBCD) (1:6)

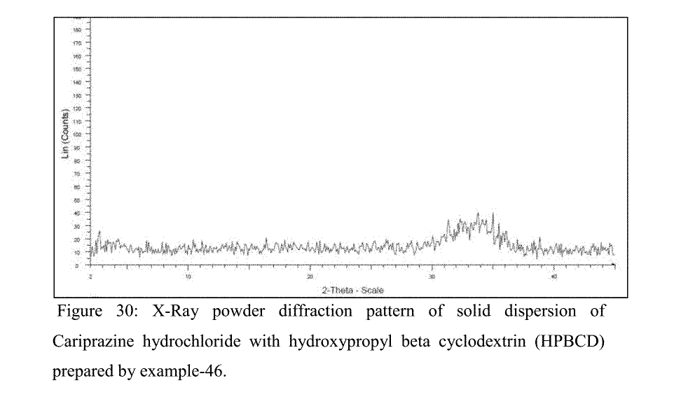

1.0 g of Cariprazine hydrochloride (1) was dissolved in 280 mL methanol and 6.0 g of hydroxypropyl beta cyclodextrin (HPBCD) was added and stirred for 15-30 minutes at 25-35° C. to form a clear solution. The clear solution was filtered, and the filtrate was distilled off below 65° C. under high vacuum and dried under vacuum to obtain solid dispersion of Cariprazine hydrochloride (1) with hydroxypropyl beta cyclodextrin (HPBCD). Yield: 93.1%; XRD: FIG. 30

### Examples-47: Preparation of Solid Dispersion of Brexpiprazole with Hydroxypropyl-β-Cyclodextrin (HPBCD) (1:3)

1.0 g of Brexpiprazole of formula (1) was dissolved in a mixture of 1:1 ratio of 40 mL dichloromethane and methanol at 25-35° C. To the reaction mass 3.0 g of hydroxypropyl-β-cyclodextrin (HPBCD) was added, stirred for 30 minutes to form a clear solution and filtered. The filtrate so obtained was taken into a Buchi flask, the solvent was evaporated completely under vacuum at 55-65° C. and dried at 45-50° C. to get the solid dispersion of Brexpiprazole with hydroxypropyl-β-cyclodextrin (HPBCD). Yield: 91%; XRPD: FIG. 31.

### Example-48: Preparation of Solid Dispersion of Eluxadoline with 1-Cyclodextrin (1:1)

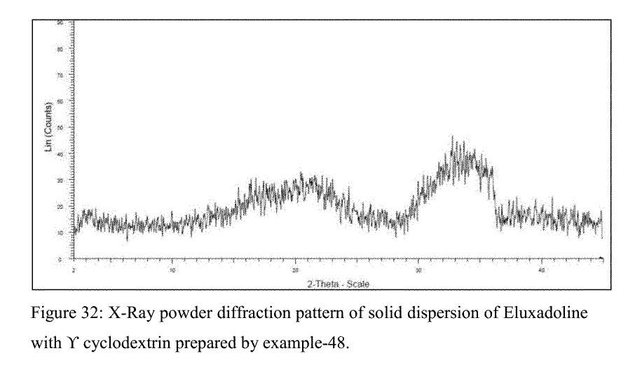

1.0 g of Eluxadoline and 1.0 g of Υ-cyclodextrin were suspended in 100 mL methanol at 25-30° C. and stirred for 15 minutes. The reaction mixture was then heated to 50-65° C. for 1 hr. The solvent was then removed completely by distillation to obtain solid dispersion of Eluxadoline with Υ-cyclodextrin. Yield: 80%; XRD: FIG. 32.

### Examples-49: Preparation of Solid Dispersion of Brexpiprazole with Maltodextrin (1:3)

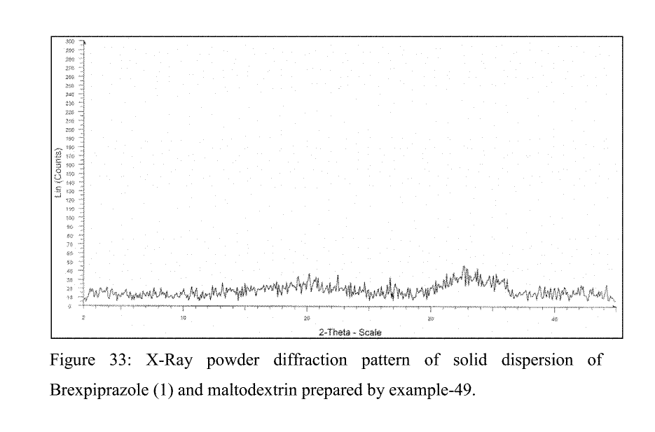

1.0 g of Brexpiprazole of formula was suspended in 20 mL of dichloromethane, a solution of 3.0 g of maltodextrin in a mixture of 1:1 ratio of 80 mL methanol and dichloromethane was added at 25-35° C. The reaction was heated to about 35-40° C. for 30 minutes and filtered. The clear solution was taken into a Buchi flask and evaporated below 45° C. and dried at 55-60° C. to get the title compound. Yield: 96%; XRD: FIG. 33.

### Example-50: Preparation of Solid Dispersion of Eluxadoline with Hydroxy Propyl Methyl Cellulose (HPMC) (1:1)

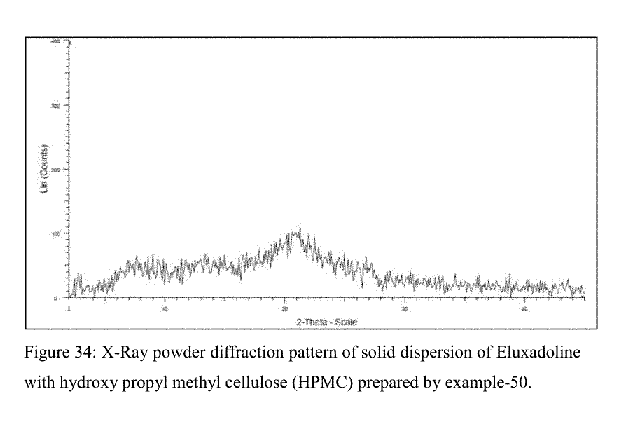

1.0 g of Eluxadoline and 1.0 g of hydroxy propyl methyl cellulose (HPMC) were suspended in 150 mL methanol at 25-30° C. The reaction mixture was then heated to 50-65° C. and stirred for 1 hr to form a clear solution. The solvent was then removed completely by distillation to obtain solid dispersion of Eluxadoline with hydroxy propyl methyl cellulose (HPMC). Yield: 81%; XRD: FIG. 34.

### Example-51: Preparation of Solid Dispersion of Eluxadoline with Hydroxy Propyl Methyl Cellulose (HPMC) (1:1.5)

1.0 g of Eluxadoline and 1.5 g of hydroxy propyl methyl cellulose (HPMC) were suspended in 100 mL methanol at 25-30° C. and stirred for 10 minutes. The reaction mixture was then heated to 50-65° C. to form a clear solution. The solvent was then removed completely by distillation to obtain solid dispersion of Eluxadoline with hydroxypropyl cellulose (HPC). Yield: 85%;

### Example-52: Preparation of Solid Dispersion of Ribociclib Succinate with Hydroxypropyl Methyl Cellulose (HPMC)(1:1)

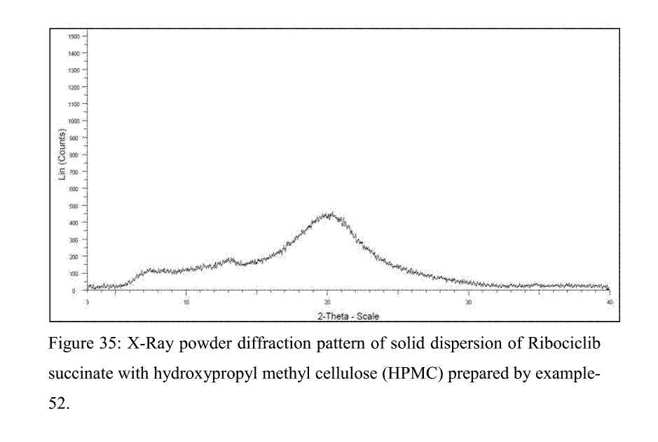

1.0 g of Ribociclib succinate and 1.0 g of hydroxypropyl methyl cellulose (HPMC) were suspended in a mixture of 20 mL methanol and 20 mL dichloromethane and stirred for 15 minutes at 25-30° C. The reaction mixture was heated to 50-65° C. till clear solution was formed. The solvent was distilled off using rotary evaporator under vacuum at 50-65° C. to obtain solid dispersion of Ribociclib succinate with hydroxypropyl methyl cellulose (HPMC). Yield: 74%; XRD: FIG. 35.

### Example-53: Preparation of Solid Dispersion of Ribociclib Succinate with Hydroxypropyl Methyl Cellulose (HPMC) (1:2)

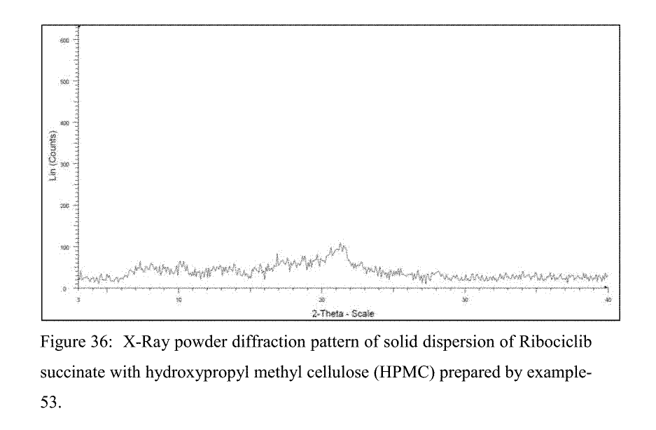

1.0 g of Ribociclib succinate and 2.0 g of hydroxypropyl methyl cellulose (HPMC) were suspended in a mixture of 160 mL methanol and stirred for 30 minutes at 25-30° C. to form a thick viscous mass. The thick viscous mass was then heated to 50-65° C. and stirred for 30 minutes to form a clear solution. The solvent was distilled off using rotary evaporator under vacuum at 50-65° C. to obtain solid dispersion of Ribociclib succinate with hydroxypropyl methyl cellulose (HPMC). Yield: 75%; XRD: FIG. 36.

### Example-54: Preparation of Solid Dispersion of Cariprazine Hydrochloride with Hydroxypropyl Methyl Cellulose (HPMC) (1:1)

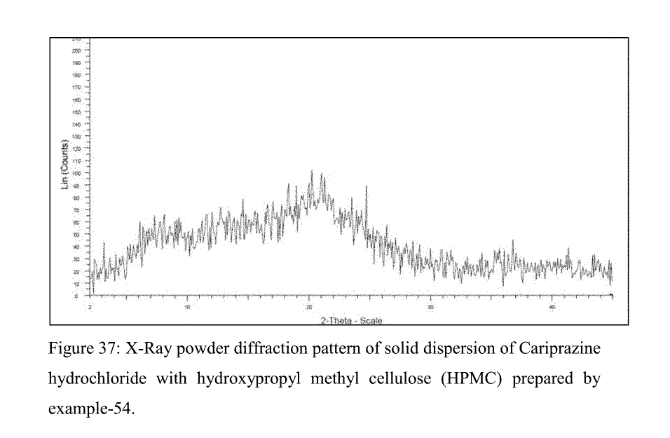

1.0 g of Cariprazine hydrochloride was dissolved in 260 mL methanol and 1.0 g of hydroxypropyl methyl cellulose (HPMC) was added at 25-30° C. The reaction mixture was heated to 55-60° C. and stirred for 15-30 minutes to form a clear solution and filtered to remove any particulate matter. The filtrate was distilled off below 65° C. under high vacuum and dried to obtain solid dispersion of Cariprazine hydrochloride with hydroxypropyl methyl cellulose (HPMC). Yield: 83%; XRD: FIG. 37.

### Example-55: Preparation of Solid Dispersion of Cariprazine Hydrochloride with Hydroxy Propyl Methyl Cellulose Acetate Succinate (HPMC-AS) (1:1)

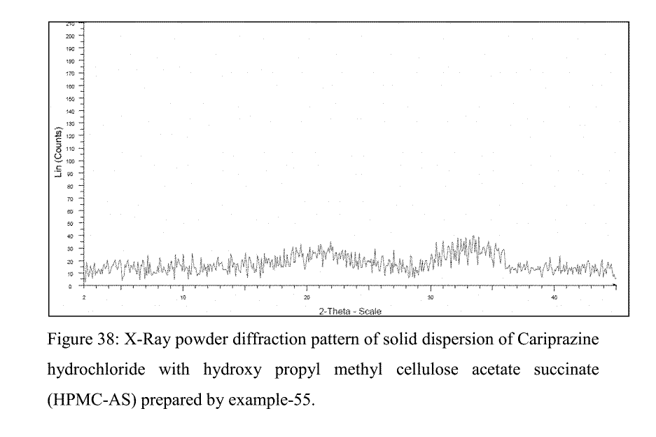

1.0 g of Cariprazine hydrochloride was dissolved in 260 mL methanol and 1.0 g hydroxy propyl methyl cellulose acetate succinate (HPMC-AS) was added at 25-30° C. The reaction mixture was heated to 55-60° C. and stirred for 15-30 minutes to form a clear solution and passed through micron filter. The filtrate was distilled off below 65° C. under vacuum and dried to obtain solid dispersion of Cariprazine hydrochloride with hydroxy propyl methyl cellulose acetate succinate (HPMC-AS). Yield: 83%; XRD: FIG. 38.

### Examples-56: Preparation of Solid Dispersion of Brexpiprazole with Hydroxypropyl Methyl Cellulose (HPMC) (1:3)

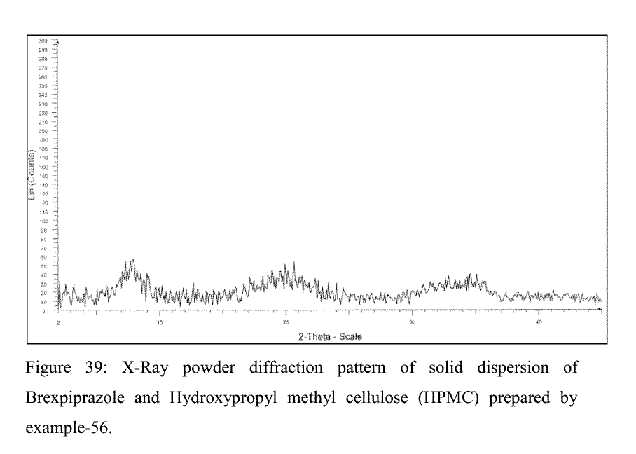

1.0 g of Brexpiprazole of formula was suspended in 20 mL of dichloromethane, a solution of 3.0 g of hydroxypropyl methyl cellulose (HPMC) in 40 mL methanol was added at 25-35° C. and passed through micron filter. The filtrate was taken into a Buchi flask and evaporated the solvent completely at 60-65° C., dried at 60-65° C. to solid dispersion of Brexpiprazole with hydroxypropyl methyl cellulose (HPMC). Yield: 98%; XRPD: FIG. 39

### Example-57: Preparation of Solid Dispersion of Eluxadoline with Hydroxypropyl Cellulose (HPC) (1:1)

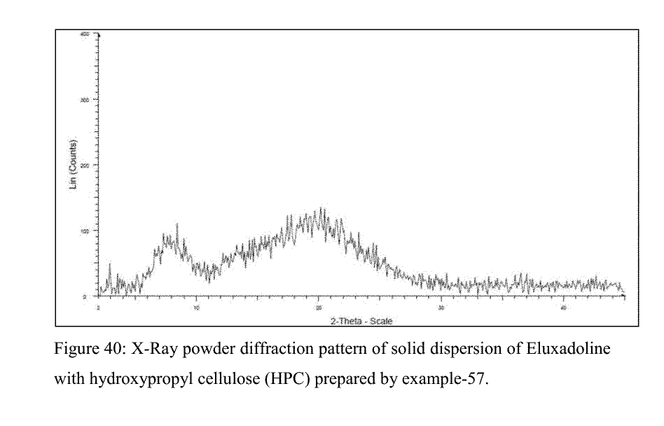

1.0 g of Eluxadoline and 1.0 g of hydroxypropyl cellulose (HPC) were suspended in 100 mL methanol at 25-30° C. The reaction mixture was then heated to 50-65° C. and stirred for 1 hr to form a clear solution. The solvent was then removed completely by distillation to obtain solid dispersion of Eluxadoline with hydroxypropyl cellulose (HPC). Yield: 86%; XRD: FIG. 40.

### Example-58: Preparation of Solid Dispersion of Eluxadoline with Hydroxypropyl Cellulose (HPC) (1:1.5)

1.0 g of Eluxadoline and 1.5 g of hydroxypropyl cellulose (HPC) were suspended in 100 mL methanol at 25-30° C. and stirred for 15 minutes. The reaction mixture was then heated to 50-65° C. to form a clear solution. The solvent was then removed completely by distillation to obtain solid dispersion of Eluxadoline with hydroxypropyl cellulose (HPC). Yield: 82%;

### Example-59: Preparation of Solid Dispersion of Ribociclib Succinate with Hydroxypropyl Cellulose (HPC) (1:2)

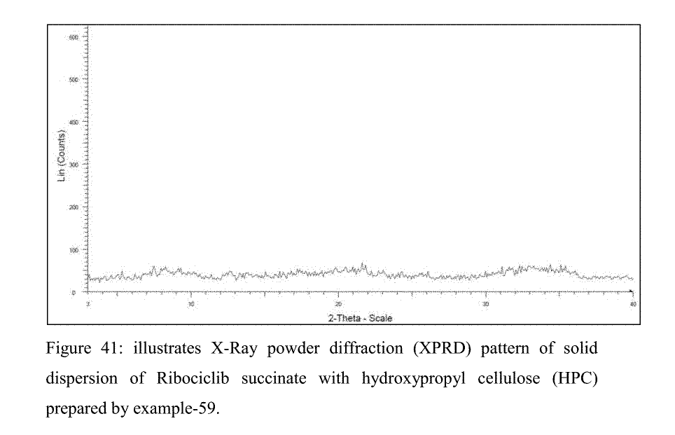

1.0 g of Ribociclib succinate and 2.0 g of hydroxypropyl cellulose (HPC) were suspended in 160 mL methanol and stirred for 15 minutes at 25-30° C. to form a thick viscous mass. The thick viscous mass was then heated to 50-65° C. and stirred for 15 minutes to form a clear solution. The solvent was distilled off using rotary evaporator under vacuum at 50-65° C. to obtain solid dispersion of Ribociclib succinate with hydroxypropyl cellulose (HPC). Yield: 72%; XRD: FIG. 41.

### Examples-60: Preparation of Solid Dispersion of Brexpiprazole with Hydroxypropyl Cellulose (HPC)

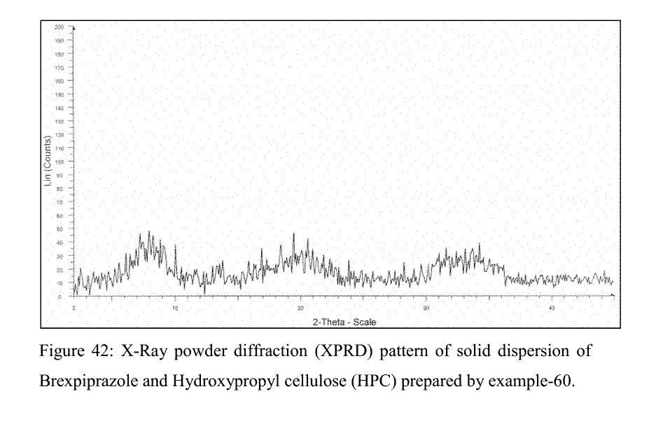

1.0 g of Brexpiprazole was suspended in 20 mL of dichloromethane, a solution of 3.0 g of hydroxypropyl cellulose (HPC) in a mixture of 1:1 ratio of 160 mL dichloromethane and methanol was added at 25-35° C. and passed through micron filter. The filtrate was taken into a Buchi flask and evaporated the solvent completely below 60° C. and dried at 55-60° C. to get solid dispersion of Brexpiprazole with hydroxypropyl cellulose (HPC). Yield: 90%; XRPD: FIG. 42.

### Example-61: Preparation of Solid Dispersion of Cariprazine Hydrochloride with Hydroxypropyl Cellulose (HPC) (1:1)

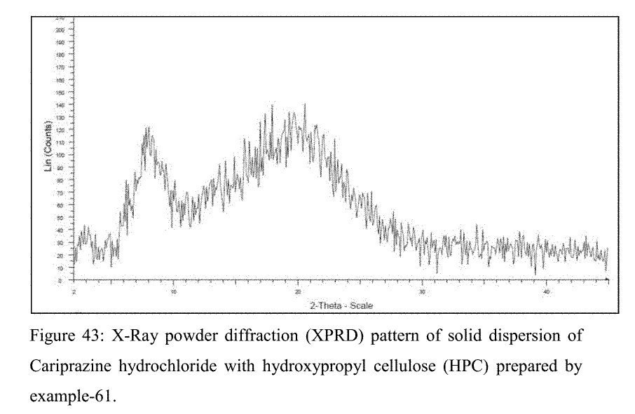

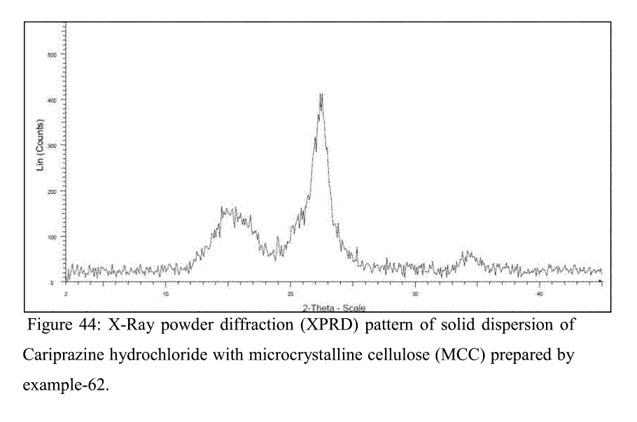

1.0 g of Cariprazine hydrochloride was dissolved in 260 mL methanol and 1.0 g of hydroxypropyl cellulose (HPC) was added at 25-30° C. The reaction mixture was heated to 55-60° C. and stirred for 15-30 minutes to form a clear solution and filtered to remove any particulate matter. The filtrate was distilled off below 65° C. under high vacuum and dried to obtain solid dispersion of Cariprazine hydrochloride with hydroxypropyl cellulose (HPC). Yield: 86%; XRD: FIG. 43.

### Example-62: Preparation of Solid Dispersion of Cariprazine Hydrochloride with Microcrystalline Cellulose (MCC) (1:8)

1.0 g of Cariprazine hydrochloride was dissolved in 800 mL methanol and 8.0 g microcrystalline cellulose (MCC)) was added at 25-30° C. The reaction mixture was heated to 55-60° C. and stirred for 15-30 minutes to form a hazy solution. The hazy solution was distilled off below 65° C. under high vacuum and dried to obtain solid dispersion of Cariprazine hydrochloride with microcrystalline cellulose (MCC). Yield: 96%; XRD: figure

### Example-63: Preparation of Solid Dispersion of Eluxadoline with Polyvinylpyrrolidone (PVP) (1:1)

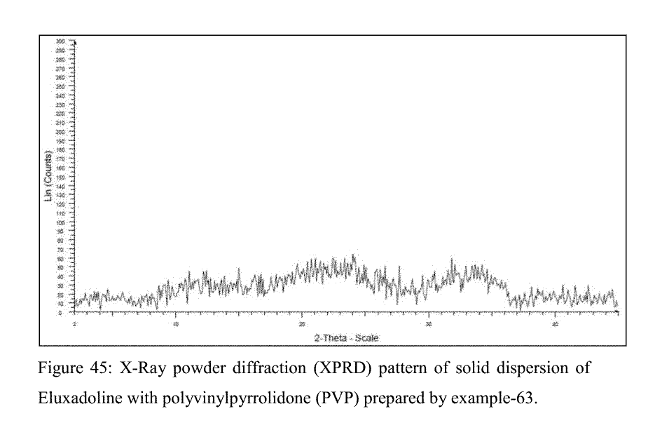

1.0 g of Eluxadoline and 1.0 g of polyvinylpyrrolidone (PVP) were suspended in 100 mL of methanol at 25-30° C. The reaction mixture was then heated to 50-65° C. and stirred for 1 hr to form a clear solution. The solvent was then removed completely by distillation to obtain solid dispersion of Eluxadoline with polyvinylpyrrolidone (PVP). Yield: 83%; XRD: FIG. 45.

### Example-64: Preparation of Solid Dispersion of Eluxadoline with Polyvinylpyrrolidone (PVP) (1:1.5)

1.0 g of Eluxadoline and 1.5 g of polyvinylpyrrolidone (PVP) were suspended in 100 mL methanol at 25-30° C. The reaction mixture was then heated to 50-65° C. and stirred for 1 hr to form a clear solution. The solvent was then removed completely by distillation to obtain solid dispersion of Eluxadoline with polyvinylpyrrolidone (PVP). Yield: 80%;

### Example-65: Preparation of Solid Dispersion of Cariprazine Hydrochloride with Polyvinylpyrrolidone (PVP K-30) (1:1)

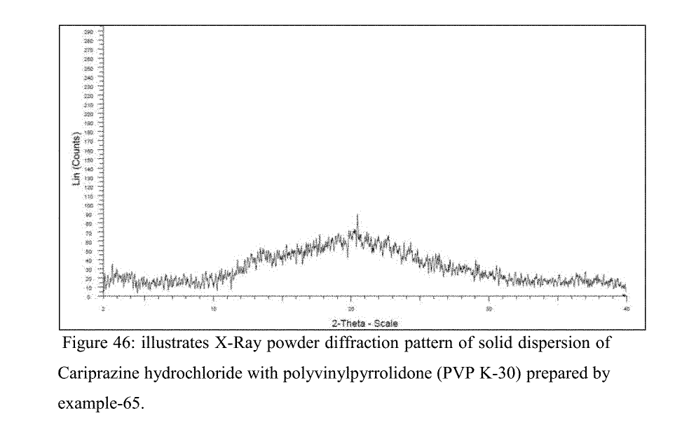

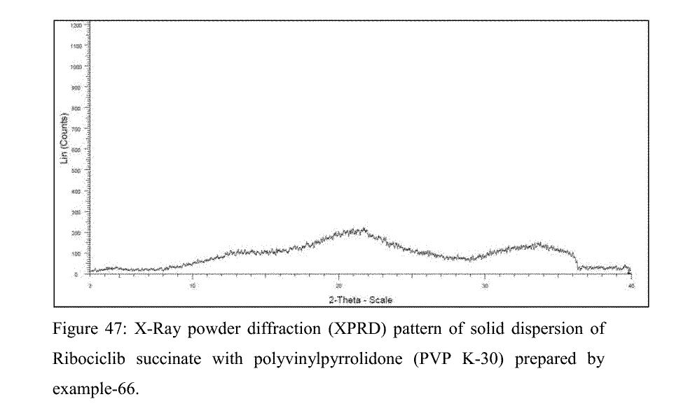

1.0 g of Cariprazine hydrochloride was dissolved in 200 mL methanol and 1.0 g of polyvinylpyrrolidone (PVP K-30) was added and stirred for 15-30 minutes at 25-35° C. to form a clear solution and passed through micron filter. The filtrate was distilled off below 65° C. under high vacuum and dried under high vacuum to obtain solid dispersion of Cariprazine hydrochloride with polyvinylpyrrolidone (PVP K-30). Yield: 83%; XRD: FIG. 46.

### Example-66: Preparation of Solid Dispersion of Ribociclib Succinate with Polyvinylpyrrolidone (PVP K-30) (1:1)

1.0 g of Ribociclib succinate and 1.0 g of polyvinylpyrrolidone (PVP K-30) were suspended in a mixture of 20 mL methanol and 20 mL dichloromethane and stirred for 15 minutes at 25-30° C. The reaction mixture was then heated to 50-65° C. and stirred for 15 minutes to form a clear solution. The solvent was then removed using rotary evaporator at 50-65° C. to obtain solid dispersion of Ribociclib succinate with polyvinylpyrrolidone (PVP K-30). Yield: 79%; XRD: 47.

### Example-67: Preparation of Solid Dispersion of Rucaparib Camsylate with Polyvinylpyrrolidone-K-30 (PVP-K-30) (2:1)

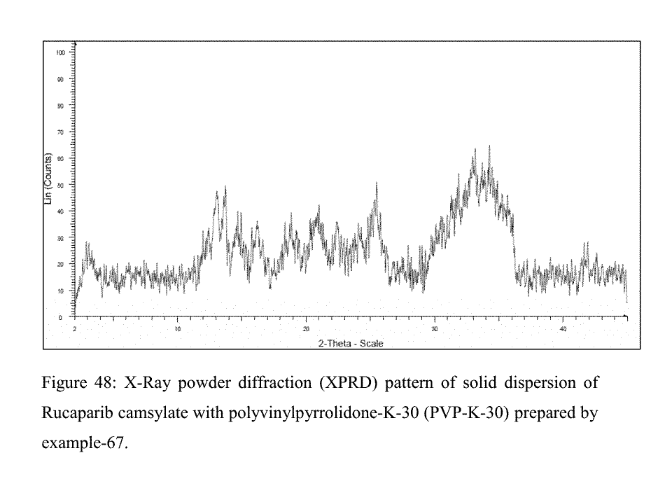

2.0 g of Rucaparib camsylate was suspended in 40 mL of methanol and 1.0 g of polyvinylpyrrolidone-K-30 (PVP-K-30) was added at 25-30° C. The reaction mixture was then heated at 55-60° C. and stirred for 15-30 minutes. 4 mL of water was added to the reaction mass, stirred for 15-30 minutes at 55-60° C. to form a clear solution and filtered. The filtrate was distilled under vacuum and the residue dried under vacuum below 60° C. to obtain solid dispersion of Rucaparib camsylate with polyvinyl pyrrolidone-K-30 (PVP-K-30). Yield: 86%; XRD: FIG. 48.

### Examples-68: Preparation of Solid Dispersion of Brexpiprazole with Polyvinylpyrrolidone K-30 (PVP K-30) (1:3)

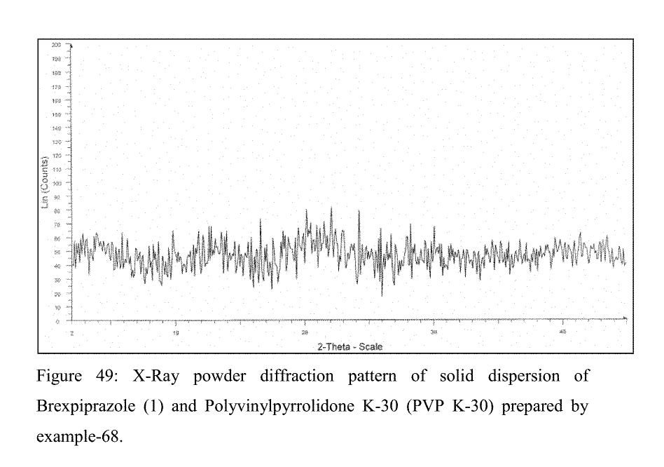

1.0 g of Brexpiprazole of formula was dissolved in a mixture of 1:1 ratio of 40 mL dichloromethane and methanol at 25-35° C. To the reaction mass 3.0 g of Polyvinyl pyrrolidone K-30 (PVP K-30) was added, stirred for 30 minutes to form a clear solution and passed through micron filter. The clear solution was taken into a Buchi flask and evaporated completely at 55-65° C. and dried at 45-50° C. to get solid dispersion of Brexpiprazole and Polyvinylpyrrolidone K-30 (PVP K-30). Yield: 90%; XRPD: FIG. 49.

### Example-69: Preparation of Solid Dispersion of Cariprazine Hydrochloride with Sulfobutylether-β-Cyclodextrin (SBECD) (1:6)

2.5 g of Cariprazine hydrochloride was dissolved in 1000 mL of methanol and 15.0 g of sulfobutylether-β-cyclodextrin (SBECD) was added at 25-30° C. The reaction mixture was then heated at 40-45° C. and stirred for 15-30 minutes to form a clear solution. The clear solution was filtered to remove any particulate matter. The filtrate was then spray dried at below mentioned parameters to obtain solid dispersion of Cariprazine hydrochloride with sulfobutylether-β-cyclodextrin (SBECD). Yield: 60.5%.

### Example-70: Preparation of Solid Dispersion of Brexpiprazole with Hydroxypropyl-β-Cyclodextrin (HPBCD) (1:3)

8.0 g of Brexpiprazole of formula and 24.0 g of hydroxypropyl-β-cyclodextrin (HPBCD) were dissolved in a mixture of 250 mL dichloromethane and 150 mL methanol at 25-35° C. The reaction mixture was passed through micron filter. The clear solution was then spray dried at below mentioned parameters to obtain the title compound. Yield: 50%.

### Example-71: Preparation of Solid Dispersion of Cariprazine Hydrochloride with Sulfobutylether-β-Cyclodextrin (SBECD) (1:6)

15.0 g of Cariprazine hydrochloride was dissolved in 2500 mL of methanol and 90.0 g of sulfobutylether-β-cyclodextrin (SBECD) was added at 25-30° C. The reaction mixture was then heated at 40-45° C. and stirred for 15-30 minutes to form a clear solution. The clear solution was passed through micron filter. The filtrate was then dried by Agitated Thin Film Drier (ATFD) to obtain solid dispersion of Cariprazine hydrochloride with sulfobutylether-3-cyclodextrin (SBECD). Yield: 50%.

### Examples-72: Preparation of Solid Dispersion of Brexpiprazole with Hydroxypropyl-β-Cyclodextrin (HPBCD) (1:3)

8.0 g of Brexpiprazole of formula was dissolved in a mixture of 250 mL dichloromethane and 150 mL methanol at 25-35° C., 24.0 g of hydroxypropyl-β-cyclodextrin (HPBCD) was added and filtered to make the solution particle free. The clear solution was then dried by agitated thin film drier method (ATFD) to obtain the title compound. Yield: 17.3%.

### Example-73: Preparation of Solid Dispersion of Crisaborole with Hydroxypropyl Methyl Cellulose (HPMC) (1:1)

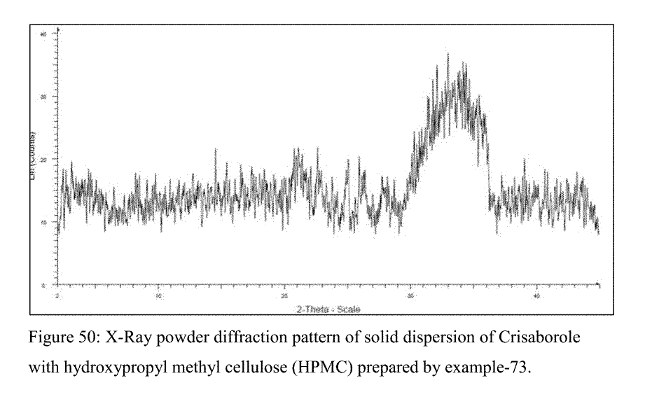

1.0 g of Crisaborole was dissolved in 5 volumes of isopropyl alcohol. The reaction mixture was stirred for 5-10 minutes at 25-30° C. to form a clear solution. 1.0 g of hydroxypropyl methyl cellulose (HPMC) was added to the clear solution and heated to 60-65° C. The reaction mixture was stirred for 1 hr at 60-65° C. and the solvent was distilled off under vacuum. To the solid, 7 volumes of cyclohexane was added and heated for 30 min at 70-75° C. The reaction mixture was cooled for 1 hr at 25-30° C. Then, reaction mixture was filtered and washed the solid with 1 volume of cyclohexane. The solid so filtered was dried under vacuum to obtain solid dispersion of Crisaborole with hydroxypropyl methyl cellulose. Yield: 1.6 g; XRD: FIG. 50.

### Example-74: Preparation of Solid Dispersion of Crisaborole with Hydroxypropyl Methyl Cellulose (HPMC) (1:3)

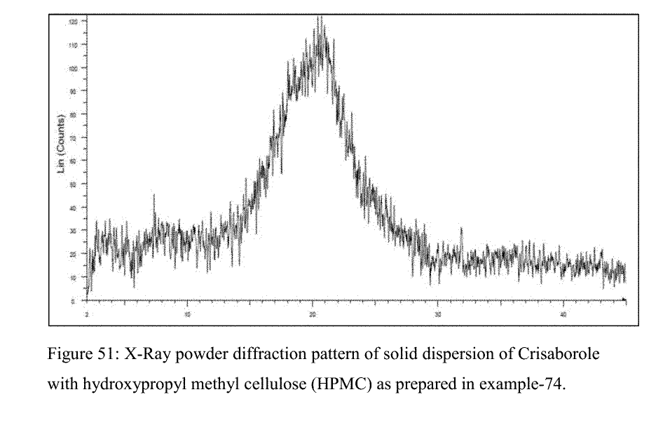

5.0 g of Crisaborole was dissolved in 5 volumes of isopropyl alcohol. The reaction mixture was stirred for 5-10 minutes at 25-30° C. to form a clear solution. 15.0 g of hydroxypropyl methyl cellulose (HPMC) was then added to the clear solution and heated to 60-65° C. The reaction mixture was stirred for 1 hr at 60-65° C. and the solvent was distilled off under vacuum. To the solid obtained, 7 volumes of cyclohexane was added and heated for 30 min at 70-75° C. The reaction mixture was cooled for 1 hr at 25-30° C., filtered and washed with 1 volume of cyclohexane. The solid so filtered was dried under vacuum to obtain solid dispersion of Crisaborole with hydroxypropyl methyl cellulose. Yield: 17.0 g; XRD: FIG. 51

### Example-75: Preparation of Solid Dispersion of Crisaborole with Hydroxypropyl Cellulose (HPC) (1:1)

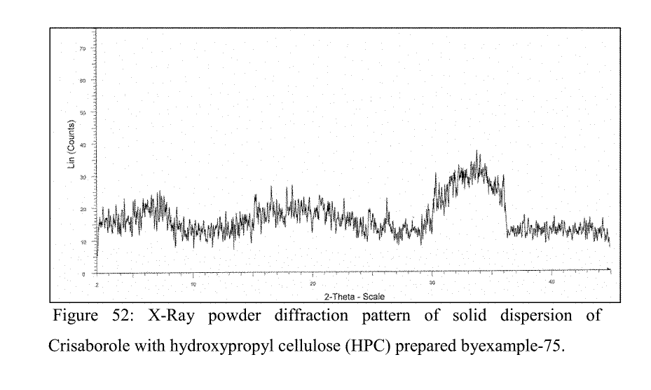

1.0 g of Crisaborole was dissolved in 5 volumes of isopropyl alcohol. The reaction mixture was stirred for 5-10 minutes at 25-30° C. to form a clear solution. 1.0 g of hydroxypropyl cellulose (HPC) was added to the clear solution and heated to 60-65° C. The reaction mixture was stirred for 1 hr at 60-65° C. and the solvent was distilled off under vacuum. 7 volumes of cyclohexane were added to the solid and heated for 30 min at 70-75° C. The reaction mixture was cooled for 1 hr at 25-30° C. Then, the precipitated solid was filtered and washed with 1 volume of cyclohexane. The solid so filtered was dried under vacuum to obtain solid dispersion of Crisaborole (I) with hydroxypropyl cellulose (HPC). Yield: 1.9 g; XRD: FIG. 52.

### Example-76: Preparation of Solid Dispersion of Crisaborole with Polyvinylpyrrolidone (PVP) (1:1.5)

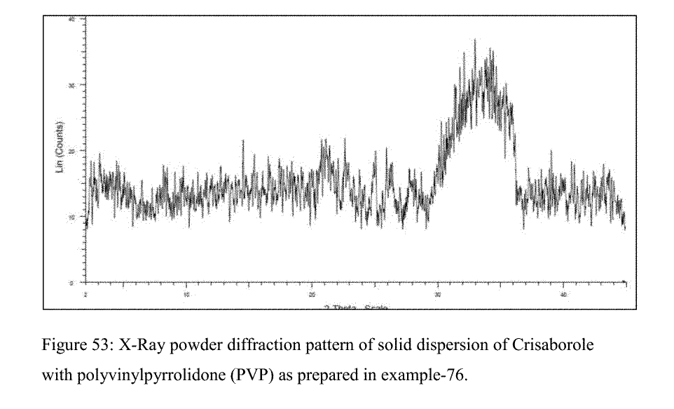

1.0 g of Crisaborole was dissolved in 10 volumes of isopropyl alcohol and stirred for 5-10 minutes at 25-30° C. to form a clear solution. 1.5 g of poly vinyl pyrrolidone (PVP) was added to the clear solution and heated for 1 hr at 60-65° C. The solvent was removed by distillation under vacuum, then cooled to 25-30° C. and the solid was treated with 7 volumes of cyclohexane. The reaction mixture was then heated for 30 min at 70-75° C., cooled for 1 hr at 25-30° C. and filtered. The precipitated solid was washed with 1 volume of cyclohexane and dried under vacuum to obtain solid dispersion of Crisaborole with of polyvinylpyrrolidone (PVP). Yield: 0.9 g; XRD: FIG. 53.

### Example-77: Preparation of Amorphous Ribociclib Succinate by Spray Drying

Method 5.0 g of Ribociclib succinate was suspended in a mixture of 40 mL methanol and 40 mL dichloromethane and stirred for 15 minutes at 25-30° C. The reaction mixture was then heated to 50-65° C. and stirred for 15 minutes to form a clear solution. The clear solution was then spray dried to obtain solid form of Ribociclib succinate. Yield: 83%;

We claim:

1. A process for preparing a solid dispersion comprising:
g) providing a solution of active pharmaceutical ingredient in a solvent or mixture of solvents;
h) adding pharmaceutically acceptable excipient to step a) solution;
i) optionally, heating the reaction mixture to a suitable temperature;
j) optionally, cooling the reaction mixture to a suitable temperature;
k) removing the solvent by a suitable method selected from a group comprising of solvent evaporation, lyophilization, spray drying, agitated thin film drying (ATFD), air tray drying, cooling the solvent, adding anti-solvent to the reaction mixture and combination thereof; and
l) isolating solid dispersion of active pharmaceutical ingredient and excipient in an amorphous form.

2. The process as claimed in claim 1, wherein the active pharmaceutical ingredient is selected from a group comprising of Tipiracil hydrochloride, Brexpiprazole, Cariprazine hydrochloride, Ribociclib succinate, Rucaparib camsylate, Eluxadoline and Crisaborole

3. The process as claimed in claim 1, wherein the excipient is selected from sulfobutylether-β-cyclodextrin SBECD, hydroxypropyl beta cyclodextrin (HPBCD), Υ-cyclodextrin, maltodextrin; hydroxy propyl methyl cellulose (HPMC), hydroxy propyl methyl cellulose acetate succinate (HPMC-AS), hydroxypropyl cellulose (HPC), microcrystalline cellulose (MCC); polyvinylpyrrolidone (PVP), pyrrolidone K-30 (PVP K-30) and lactose or mixtures thereof.

4. The process as claimed in claim 1, wherein the solvent in step a) is selected from a group comprising of water, methanol, ethanol, isopropyl alcohol, isobutyl alcohol, dimethyl sulfoxide or acetone, acetonitrile, nitromethane, 1,4-dioxane, diethyl ether, dichloromethane, ethyl acetate, N, N-dimethylformamide, methyl tertiary butyl ether hexane, butyl acetate cyclohexane, toluene, tetrahydrofuran or mixtures thereof.

5. The process as claimed in claim 1, wherein the active pharmaceutical ingredient in step a) can be crystalline, amorphous, salt, solvate or free base.

6. The process as claimed in claim 1, wherein the ratio of active pharmaceutical ingredient to the excipient range from of 1:0.3 to 1:20 (w/w).

7. A process for preparing a solid dispersion of Eliglustat hemitartrate, comprising:
a) providing a solution of Eliglustat hemitartrate in a solvent or mixture of solvents;
b) adding suitable pharmaceutically acceptable excipient to step a) solution, wherein the said excipient is cyclodextrin derivative;
c) optionally, heating the reaction mixture to a suitable temperature;
d) optionally, cooling the reaction mixture to a suitable temperature;
e) removing the solvent by a suitable method selected from solvent evaporation lyophilization or spray drying or agitated thin film drying (ATFD) or air tray drying or cooling the solvent or adding anti-solvent to the reaction mixture or combination thereof; and
f) isolating s solid dispersion of Eliglustat hemitartrate and excipient in an amorphous form.

8. The process as claimed in claim 7, wherein the cyclodextrin derivative is selected from a group comprising of sulfobutylether-β-cyclodextrin (SBECD), hydroxypropyl beta cyclodextrin (HPBCD) and Υ-cyclodextrin.

9. The process as claimed in claim 7, wherein the ratio of active pharmaceutical ingredient to the excipient ranges from of 1:0.3 to 1:20 (w/w).

10. The process as claimed in claim 7, wherein the solvent used in step a) is selected from a group comprising of water, methanol, ethanol, isopropyl alcohol, isobutyl alcohol, dimethyl sulfoxide or acetone, acetonitrile, nitromethane, 1,4-dioxane, diethyl ether, dichloromethane, ethyl acetate, N, N-dimethylformamide, methyl tertiary butyl ether hexane, butyl acetate cyclohexane, toluene, tetrahydrofuran or mixtures thereof.
